(19) United States
(12) Patent Application Publication
(10) Pub. No.: US 2026/0258161 A1
(43) Pub. Date: Sep. 3, 2026

(54) METHODS FOR TRANSFORMING POLYSACCHARIDES TO CROSS-LINKABLE POLYSACCHARIDES AND PRODUCTS THEREOF

(71) Applicant: University of Ottawa, Ottawa (CA)

(72) Inventors: Andrew PELLING, Ottawa (CA); Maxime LEBLANC LATOUR, Ottawa (CA); Cesar Augusto TISCHER, Ottawa (CA)

(21) Appl. No.: 18/993,274

(22) PCT Filed: Jul. 19, 2023
(86) PCT No.: PCT/CA2023/050970

§ 371 Date: Jan. 10, 2025

Related U.S. Application Data
(60) Provisional application No. 63/390,420, filed on Jul. 19, 2022.

(51) Int. Cl. C08B 15/10; A61K 8/73; A61L 31/10; A61Q 3/02; C08B 35/00; C09D 101/02; C09D 103/14

(52) U.S. Cl. CPC C08B 15/10; A61K 8/731; A61K 8/732; A61L 31/10; A61Q 3/02; C08B 35/00; C09D 101/02; C09D 103/14

(57) ABSTRACT

A method of transforming a polysaccharide into a cross-linkable polysaccharide is described which comprises providing a solution comprising a solubilized polysaccharide, converting the polysaccharide into an amino-polysaccharide by replacing at least one reactive group of the polysaccharide with an amine group, thereby creating the amino-polysaccharide; and crosslinking the amino-polysaccharide with methacrylate, thereby creating a cross-linkable polysaccharide. Also described is a method of transforming a cross-linkable polysaccharide into a light cross-linkable polysaccharide. Additionally, there is described a method of transforming a light cross-linkable polysaccharide into resin. Additionally, a method of transforming a polysaccharide into a cross-linkable polysaccharide via an intermediary molecule is provided. Lastly, a method of preparing a protective coating using the cross-linkable polysaccharide is provided.

### FIELD OF THE INVENTION

The present invention generally relates to the transformation of polysaccharides into cross-linkable polysaccharides. More specifically, the present invention relates to methods for the transformation of polysaccharides and to the cross-linkable polysaccharides, UV cross-linkable polysaccharides and/or resins obtained thereof.

### BACKGROUND OF THE INVENTION

Cellulose has gained interest in recent years for many applications, such as paper manufacturing, packaging, plastic alternative, electronics, consumables, and medical engineering. Cellulose, along with hemicellulose, lignin, and pectin, forms lignocellulosic materials that constitute plants cell walls and its biomass represents the most abundant available natural polymer. In plants, lignocellulosic material forms a complex architecture that can consist of interconnected pores, channels, or alternating layers. These features, which differ from plant type to plant type, can be conserved by removing the cellular components using chemical treatments, thereby leaving only the lignocellulosic cell wall as an empty scaffold. Alternatively, cellulose can be extracted from trees and plants to form fibers, or it can be synthesised by bacteria in pure form. Chemically, cellulose is a polymer consisting of linearly repeating glucose units forming (1→4) glycosidic bonds whereas individual cellulose chains can be linked to one another via hydrogen bonds. On a microscopic level, cellulose chains form fibrillar bundles ranging in the few hundreds micrometers. These fibrillar bundles can be chemically treated into smaller fibrillar structures to form nanocellulose. Nanocellulose fibrils, or cellulose nanofibrils (CNFs), have been proven to be biocompatible and used in many bioengineering applications. Recently, the use of cellulose in applications in the field bone tissue engineering (BTE) has gained interest.

Scaffolds for traditional tissue engineering are primarily harvested from patient bodies, cadavers, animals, synthetic or plant/bacterial polymers. None of the above are controllable at the micro/nanoscale. Three-dimensional printing methods exist for controlling the micro/nano structure, but are only available for a few synthetic polymers (e.g, polyethylene glycol diacrylate) and natural polymers (e.g. silk). Disadvantages of those polymers include degradation by-products, extensive protocols to derive the material, and limited water solubility. Moreover, cellulose is not dissolvable in water, which limits its ability to chemically functionalize with biological (or water soluble) functional groups. Despite these obstacles, cellulose remains a highly useful material in biomaterials research as it is renewable, widely available and low cost.

Pectins are a group of acidic heteropolysaccharides typically found in primary cell walls of terrestrial plants (https://doi.org/10.1016/j.carbpol.2017.03.058) and are the second most abundant polysaccharides found in plant cell walls. Pectins are mostly composed of α-d-galacturonic acid residues and a variety of neutral sugars such as rhamnose (https://www.sciencedirect.com/topics/biochemistry-genetics-and-molecular-biology/rhamnose), galactose (https://www.sciencedirect.com/topics/biochemistry-genetics-and-molecular-biology/galactose) and arabinose (https://www.sciencedirect.com/topics/biochemistry-genetics-and-molecular-biology/arabinose). The main chain in pectin is a linear galacturonan, with unsubstituted regions known as smooth regions interposed by residues substituted with heteropolysaccharides and two types of rhamnogalacturonans (RG): RG-I and RG-II (https://doi.org/10.1016/j.ijbiomac.2017.03.029).

Despite pectin's solubility in water, the formation of pectin gels only occur when heated from 40° C. up to 105° C. through a reaction driven by the presence of a methoxy which leads a pectin solution to jellify. The formation of pectin gels is pH-dependent and requires calcium ions (Ca²⁺) to crosslink the galacturonan linear chains and generate gels that are highly suitable for extrusion as used in additive printing. However, pectin gels are not strong enough to be manipulated as an artifact or as a primer for coating purposes.

A major challenge in the preparation of cross-linkable polysaccharides such as cellulose is their low water solubility as they are often mostly insoluble. Different approaches have been developed to rectify the solubility problem. For example, starting the preparation of cross-linkable polysaccharides with a modified water-soluble polysaccharide such as methylcellulose sidesteps the water solubility problem. Other approaches include modifying certain unprotected groups found in several polymers derived from cellulose such as —OH groups found on the backbone of cellulose-derived polymers but excluding those —OH groups found within other functional groups, for example, the —OH groups comprised within ester groups. These —OH groups can be substituted with a covalently bound photo cross-linkable group such as methacrylate while keeping the original chemistry of the cellulose-derived polymer. For example, —OH groups in methylcellulose may be substituted with methacrylate without modifying the methyl group. This approach limits the possibility of chemically modifying other functional groups which would change properties of the polysaccharide.

Other approaches developed to address the low solubility of cellulose entails modifying nanocellulose to produce resins in which the nanocellulose is crosslinked with weak ionic interactions (6). Accordingly, these resins have a limited number of applications for which they can be useful.

In contrast, ionic polysaccharides such as pectin can be crosslinked with divalent cations (Ba²⁺, Ca²⁺, Mg²⁺, Zn²⁺) to produce gels. However, this approach leads to a loss of structural integrity in presence of water as the cations are released, leading to disaggregation of the gel.

Another concern is that functionalization of viscous polysaccharides consumes high amounts of energy for the removal of water (https://doi.org/10.1039/C3GC42096E) through precipitation, centrifugation and filtration.

### SUMMARY OF THE INVENTION

The shortcomings of the prior art are generally mitigated by the methods described herein.

Accordingly, there is provided herein a method of transforming a polysaccharide into a cross-linkable polysaccharide which comprises providing a solution comprising a solubilized polysaccharide, converting the polysaccharide into an amino-polysaccharide by replacing at least one reactive group of the polysaccharide with an amine group, thereby creating the amino-polysaccharide; and crosslinking the amino-polysaccharide with methacrylate, thereby creating a cross-linkable polysaccharide.

A second aspect of the invention is directed to a method of transforming a water soluble polysaccharide into a cross-linkable polysaccharide which comprises providing a solubilized polysaccharide, and crosslinking the solubilized polysaccharide to a methacrylate-containing molecule, thereby creating a cross-linkable polysaccharide.

According to a preferred embodiment, the solubilized polysaccharide is at a pH of between about 5.5 to about 10.

A third aspect of the invention is directed to method of transforming a cross-linkable polysaccharide into a light cross-linkable polysaccharide which comprises providing a solution comprising a cross-linkable polysaccharide as described above and mixing the solution with about 0.1 to 0.4% w/w or % w/v of a photo-initiator in the dark, thereby making a solution comprising the light cross-linkable polysaccharide. According to a preferred embodiment, the method further comprises storing the solution comprising the light cross-linkable cross-linkable polysaccharide in the dark.

A fourth aspect of the invention is directed to a method of transforming a light cross-linkable polysaccharide into resin which comprises providing a solution comprising a light cross-linkable polysaccharide as described above and exposing the solution to a wavelength of between about 320 nm to about 450 nm, preferably 405 nm, for between about 5 minutes to about 30 minutes.

In another aspect of the invention, a method of preparing an intermediary methacrylated-amine molecule is provided. The method involves mixing a methacrylate-containing molecule and an amine-containing molecule to prepare a reaction mixture and adding a catalyst to the reaction mixture at a temperature ranging from 20° C.-50° C. to obtain the intermediary methacrylated-amine molecule. The catalyst used could be triethylamine, N,N-diisopropylethylamine, N,N-diethylbenzylamine, or pyridine.

In another aspect of the invention, a method of transforming a polysaccharide into a cross-linkable polysaccharide is provided. The method involves preparing a mixture comprising a tosyl-polysaccharide and an intermediary methacrylated-amine molecule; and allowing the mixture to react for at least one hour at a temperature ranging from 70° C.-90° C. to obtaining the cross-linkable polysaccharide.

The invention is further directed to a cross-linkable polysaccharide obtained according to the method described above.

The invention is further directed to a light cross-linkable polysaccharide obtained according to the method described above.

The invention is further directed to a resin obtained according to the method described above.

Another aspect of the invention is directed to a use of the cross-linkable polysaccharide for preparing a light cross-linkable polysaccharide.

Another aspect of the invention is directed to a use of the light cross-linkable polysaccharide for preparing a resin.

Another aspect of the invention is directed to a method of preparing a protective coating is provided. The method involves mixing two components, component A and component B. Component A is a cross-linkable polysaccharide or a light cross-linkable polysaccharide obtained using any of the above-recited methods. Component B is a base formulation that has a diacrylate; a resin; and optionally a photo-initiator. The mixing step is followed by a step of curing the mixture by exposing it to UV light for a predetermined time, to obtain the protective coating.

Other and further aspects and advantages of the present invention will be better understood upon the reading of the illustrative embodiments about to be described or will be indicated in the appended claims, and various advantages not referred to herein will occur to one skilled in the art upon employment of the invention in practice.

### BRIEF DESCRIPTION OF THE DRAWINGS

The above and other aspects, features and advantages of the invention will become more readily apparent from the following description, reference being made to the accompanying drawings in which:

FIG. 1 shows Fourier transform infrared (FTIR) spectra of methacrylated cellulose nano fibrils (top) compared to pristine cellulose nano fibrils (bottom), according to an embodiment.

FIG. 2 shows FTIR spectra of cellulose nano fibrils, methacrylated cellulose nano fibrils and the corresponding reaction intermediates, with pristine CNF (bottom), CNF after tosylation (middle bottom), CNF after substitution with ethylenediamine (middle top) and methacrylated cellulose nano fibrils (mCNF) (top) shown in (A). methacrylated cellulose nano fibrils (mCNF) before (black) and after (red) cross-linking are shown in (B), according to an embodiment.

FIG. 3 shows ¹³C solid state NMR spectra of methacrylated cellulose nano fibrils (mCNF, top) compared to pristine cellulose nanofibrils (CNF, bottom), according to an embodiment.

FIG. 4 shows an example of a methacrylated cellulose nano fibrils resin used for UV 3D Printing with the corresponding computer-assisted models, according to an embodiment. From left to right: mCNF in solution at 0.5 g/mL; mCNF hydrogels in different shapes: Disks; 3D printed cubed; 3D printed grid, according to an embodiment.

FIG. 5 shows an example of rehydration of mCNF aerogel over 30 s: A drop of α-MEM is pipetted on the scaffold which progressively absorbs the fluid, according to an embodiment.

FIG. 6 shows an example of scanning electron microscopy micrographs of a hydrogel and an aerogel surface (Scale=100 μm, applies for both), according to an embodiment.

FIG. 7 shows an example of pore size distribution in an aerogel, from histological section images (Scale=50 μm), according to an embodiment.

FIG. 8 shows storage modulus as a function of time, with fixed sheer-stress amplitude (0.25% for 30 sec) for mCNF resin at 0.1 g/mL (bottom) and 0.5 g/mL (top) concentrations. Dotted lines represent 1 min illumination at 405 nm, according to an embodiment.

FIG. 9 shows storage modulus as a function of applied strain of mCNF at 0.1 g/mL (squares) and 0.5 g/mL (circles) concentrations, according to an embodiment.

FIG. 10 shows Young's modulus of mCNF hydrogels at 0.1 g/mL and 0.5 g/ml (Hydro 0.1 and Hydro 0.5, respectively) and mCNF aerogels at 0.1 g/mL and 0.5 g/ml (Aero 0.1 and Aero 0.5, respectively) (A), and compressive strength of hydrogels at 0.1 g/mL and 0.5 g/ml (Hydro 0.1 and Hydro 0.5, respectively) (B), according to an embodiment.

FIG. 11 illustrates MC3T3-E1 seeded aerogels after 4 weeks of incubation in either osteogenic-inducing media (OM) or α-MEM (CTRL) (top, Scale=1 mm, applies for both). Histological sections of MC3T3-E1 cell-seeded aerogels after 4 weeks of incubation in osteogenic-inducing media (OM) α-Minimum Essential Medium (MEM) (CTRL). Samples were stained with Von Kossa (VK) to highlight mineralization of the constructs (bottom, Scale=100 μm, applies for histological sections), according to an embodiment.

FIG. 12 shows Young's modulus of MC3T3-E1 seeded mCNF aerogels after 4 weeks of incubation in either osteogenic-inducing media (OM) or α-MEM (CTRL).

FIG. 13 shows Alizarin red S (ARS) staining of MC3T3-E1 seeded aerogels, after 4 weeks of incubation in either osteogenic-inducing media (OM) or α-MEM (CTRL) with calcium deposition (mineralization) of the constructs shown by a red coloration (Scale=2 mm, apply for both) (A), and quantification of mineral deposition with Alizarin Red S (ARS) staining after 4 weeks of incubation in either osteogenic-inducing media (OM) or α-MEM (CTRL) with values reported as the absorption at 405 nm (B), according to an embodiment.

FIG. 14 shows energy-dispersive X-ray spectroscopy spectra of printed mCNF squares (top) compared to MC3TE-E1-seeded squares (bottom) after four weeks in osteogenic-inducing media, according to an embodiment.

FIG. 15 shows printed cubes of hydrogel scaffolds produced with methacrylated cellulose nanofibrils resin 4 weeks after being seeded with MC3T3-E1 pre-osteoblast cells (A, B) compared with no cells seeded (C), according to an embodiment.

FIG. 16 shows bone glue produced with methacrylated cellulose nanofibrils resin under UV light for curing with the arrows showing holes filled with mCNF resin, according to an embodiment.

FIG. 17 shows GFP-3T3 cells on a resin-coated culture dish, according to an embodiment.

FIG. 18 shows aluminum (A), wood (B) and stainless steel (C) coated with mCNF resin, according to an embodiment.

FIG. 19 shows FTIR spectra of methacrylated pectin (bottom line at 4000 cm⁻¹) compared to pristine pectin (top line at 4000 cm⁻¹), according to an embodiment.

FIG. 20 shows ¹³C solid state NMR spectra of methacrylated pectin (bottom) compared to pristine pectin (top), according to an embodiment.

FIG. 21 shows an example of cross-linking of (A) a stiff disk produced with methacrylated pectin resin on a petri dish exposed to UV lamp for 5 min, (B) printed “haystack” shape using the methacrylated pectin resin by UV Digital Light Processing—DLP UV 3D printing and (C) printing a cylindrical shape using stereolithography, according to an embodiment.

FIG. 22 shows an example of a computer-assisted design of a cylindrical shape to be printed with methacrylated pectin (A), a printed methacrylated pectin cylinder (B), a stereolithography printing process (C), and cross-linking of methacrylated pectin after printing (D), according to an embodiment.

FIG. 23 illustrates a confocal microscopy image of cells grown around the printed cylinder structure depicted in FIG. 22, according to an embodiment.

FIG. 24 shows the FTIR spectrum of conversion of CNF to tosylCell where the procedure involves solvent exchange of the slurry with 99% Ethanol.

FIG. 25 shows the tosylCell obtained after solvent exchange with the CNF slurry and the reaction.

FIG. 26 shows the FTIR spectrum of preparation of methacrylated CNF (mCNF) using methacrylic anhydride i.e. the reaction of aminoCell with methacrylic anhydride.

FIG. 27 shows the dry solids of methacrylated CNF (mCNF) in the dialysis tube obtained using methacrylic anhydride.

FIG. 28 shows the dry solids of methacrylated CNF obtained using GMA i.e via reaction of aminoCell and glycidyl methacrylate in the dialysis tube.

FIG. 29 shows the scanning electron microscope image of methacrylated CNF obtained using GMA i.e via reaction of aminoCell and glycidyl methacrylate.

FIG. 30 shows the FTIR spectrum of mCNF i.e. products from the reaction of aminoCell with glycidyl methacrylate and triethylamine.

FIG. 31 shows the dried mCNF obtained following reaction of GMA with TEA catalyst.

FIG. 32 shows the FTIR spectrum of mCNF i.e products from the reaction of CNF in suspension in DMSO with methacrylic anhydride and pyridine.

FIG. 33 shows the isolated mCNF, following reaction of CNF slurry (in DMSO) with methacrylic anhydride and pyridine as a catalyst.

FIG. 34 shows the FTIR spectrum of tosylCell to mCNF (via intermediary) where the intermediary reacted at room temperature.

FIG. 35 shows the FTIR spectrum of tosylCell to mCNF (via intermediary) where the intermediary reacted at 50° C.

FIG. 36 shows the mCNF (via intermediary) generated when the intermediary is reacted at room temperature.

FIG. 37 shows the mCNF (via intermediary) generated when the intermediary is reacted at 50° C.

FIG. 38 shows the coated Medium Density fiber wood with the mCNF (via MA)-containing formulation—5 coats with 30 sec UV exposure.

FIG. 39 shows the coated Medium Density fiber wood with the mCNF (via MA)-containing formulation “scrub test”.

FIG. 40 shows the coated Medium Density fiber wood with the mCNF (via GMA)-containing formulation—5 coats with 30 sec UV exposure.

FIG. 41 shows the coated sanded wood with the mCNF-containing formulation—1 coat with 5 min UV exposure.

FIG. 42 shows the coated printed circuit board with the mCNF (via MA)-containing formulation—1 coat with 3 min UV exposure.

FIG. 43 shows the casted and cured disk made with the mCNF (via GMA)-containing formulation and 5 min UV exposure.

FIG. 44 shows UV curable nail polish formulation including mCNF (via GMA) is shown where a small coat of the formulated mixture was applied to an artificial nail with a paint brush and exposed to 405 nm UV light for 5 minutes.

### DETAILED DESCRIPTION

In view of the various challenges noted above, there is a need for a method to produce stronger, cross-linkable polysaccharides, particularly covalently linked cellulose derivatives and new methods for converting cellulose and pectin into biocompatible, water-soluble resins would thus be highly desirable. There is also a need for a method to produce ionic polysaccharides with stronger covalent linkages capable of structurally resisting the effect of water.

Another important challenge in the industry is the development of new approaches that will minimize energy consumption. Moreover, since water is becoming an increasingly scarce commodity all over the world, approaches that will lower water consumption are thus highly desirable. New methods of producing functionalized ionic heteropolysaccharides with a lower energy footprint and reduced water waste are thus highly desirable

Described herein are methods of transforming a polysaccharide into a cross-linkable molecule. Also described herein are methods of transforming a cross-linkable molecule into a light cross-linkable molecule. Methods of transforming a light cross-linkable molecule into resin are also described herein. It will be appreciated that embodiments and examples are provided for illustrative purposes intended for those skilled in the art, and are not meant to be limiting in any way.

### EMBODIMENTS

In the present invention, there is provided herein a method of transforming a polysaccharide into a cross-linkable polysaccharide which comprises providing a solution comprising a solubilized polysaccharide, converting the polysaccharide into an amino-polysaccharide by replacing at least one reactive group of the polysaccharide with an amine group, thereby creating the amino-polysaccharide; and crosslinking the amino-polysaccharide with methacrylate, thereby creating a cross-linkable polysaccharide.

According to a preferred embodiment, the reactive group is hydroxyl, methoxyl, methyl, ether, ester, carbonyl or carboxylic acid.

According to a further embodiment, the solubilized polysaccharide is obtained by heating a solution comprising the polysaccharide in a suitable solvent to between about 20° C. to about 150° C. for between about 5 minutes to about an hour.

According to a further embodiment, the solvent is a polar aprotic solvent such as a mixture of dimethylacetamide and lithium chloride, dimethyl sulfoxide (DMSO), or dimethylformamide (DMF); or an ionic liquid such as 1-butyl-3-methylimidazolium chloride (BMIMCl) or 1-allyl3-methylimidazolium chloride (AMIMCl).

According to a further embodiment, the solubilized polysaccharide is obtained by cooling a solution comprising the polysaccharide in an alkali solvent, such as a mixture of NaOH and Urea, to between about −25° C. to about 10° C. for between about 5 minutes to about an hour.

According to a further embodiment, the solubilized polysaccharide is obtained by heating a solution comprising the polysaccharide, dimethylacetamide and lithium chloride to between about 50° C. to about 150° C. for between about 5 minutes to about an hour.

According to a further embodiment, the at least one reactive group of the polysaccharide excludes hydroxyl groups directly linked to the polysaccharide backbone.

According to a further embodiment, the solubilized polysaccharide is obtained by centrifuging a solution comprising the polysaccharide and a solvent and repeating the centrifugation step in the presence of a highly concentrated alcohol. The highly concentrated alcohol can be any suitable alcohol known in the art. In a preferred embodiment, the highly concentrated alcohol is 99% ethanol.

According to a further embodiment, converting the polysaccharide into an amino-polysaccharide comprises tosylation followed by amination of the at least one reactive group.

According to a further embodiment, tosylation comprises mixing the solubilized polysaccharide with triethylamine and a tosyl-containing molecule for about 24 hours at a temperature of up to about 30° C., thereby obtaining a tosyl-polysaccharide.

According a further embodiment, tosylation comprises mixing the solubilized polysaccharide with a pyridine-ethanol solution and a tosyl-containing molecule at a low temperature for about 1 hour, thereby obtaining a tosyl-polysaccharide.

According to a further embodiment, the mixing step is followed by a step of filtering the mixture to obtain a solid precipitate. The filtration step is followed by a step of washing the solid precipitate in a highly concentrated alcohol and the washing step is followed by a step of drying the solid precipitate to obtain the tosyl-polysaccharide.

According to a further embodiment, amination comprises mixing the tosyl-polysaccharide with ethylenediamine for between about 2 hours to about 48 hours.

According to a further embodiment, crosslinking the amino-polysaccharide with methacrylate comprises dissolving the amino-polysaccharide in a solvent and mixing it with a methacrylate-containing molecule at a temperature ranging from 20° C. to 60° C.

According to a further embodiment, the mixing step comprises incubating the amino-polysaccharide with the methacrylate-containing molecule for about 1 hour to about 48 hours.

According to a further embodiment, the incubation step comprises adding a catalyst to the reaction mixture of the amino-polysaccharide and the methacrylate-containing molecule. The catalysts could be any known catalyst such as triethylamine. In an alternate embodiment, the catalysts used can be triethylamine, N,N-diisopropylethylamine, N,N-diethylbenzylamine, or pyridine

According to a further embodiment, the incubation step further comprises increasing the reaction temperature to about 60° C.-90° C. after the catalyst is added to the reaction mixture.

According to a further embodiment, the method further involves precipitating the reaction mixture using alcohol to obtain a precipitate and filtering the precipitate using any filtration technique to obtain the cross-linkable polysaccharide. In some embodiments, a vacuum filtration technique may be employed.

According to a further embodiment, the method further involves a step of cooling the precipitate or drying the precipitate to obtain the cross-linkable polysaccharide.

According to a further embodiment, tosylation comprises mixing the solubilized polysaccharide with triethylamine and a tosyl-containing molecule for about 24 hours at a temperature of up to about 30° C., thereby obtaining a tosyl-polysaccharide.

According to a further embodiment, amination comprises mixing the tosyl-polysaccharide with ethylenediamine for between about 2 hours to about 48 hours. The amine-containing molecule used could be ethylenediamine, diethylenetriamine 1,3-Diaminopropane, Putrescine, Cadaverine,1,2-Dimethylethylenediamine, Hexamethylenediamine or 1,1-Dimethylethylenediamine.

According to a further embodiment, crosslinking the amino-polysaccharide with methacrylate comprises incubating the amino-polysaccharide with a methacrylate-containing molecule for between about 1 hour to about 48 hours.

In further non-limiting embodiments, the polysaccharide is cellulose, pectin, starch, amylose, amylopectin, glycogen, gum arabic, gum ghatti, gum karaya, pullulan, β-glucans, dextran, xanthan, alginate, gellan LA, levan, hyaluronic acid or chitosan, preferably cellulose.

In another non-limiting embodiment, the amino-polysaccharide is a pyridine-polysaccharide.

In further non-limiting embodiments, the methacrylate-containing molecule is 2-hydroxyethyl methacrylate (HEMA), 2-hydroxypropyl methacrylate (HPMA), acrylamide (AAm), acrylic acid (AAc), N-isopropylacrylamide (NIPAm), methoxyl poly (ethylene glycol) (PEG) monoacrylate (mPEGMA or PEGMA), N,N′-methylenebis(acrylamide) (MBA), ethylene glycol diacrylate (EGDA), PEG diacrylate (PEGDA), methacrylic anhydride, glycidyl methacrylate or any other suitable methacrylate-containing molecule.

In another embodiment of the invention, a method of transforming a water soluble polysaccharide into a cross-linkable polysaccharide is provided. The method involves providing a solubilized polysaccharide, and crosslinking the solubilized polysaccharide with a methacrylate-containing molecule, thereby creating a cross-linkable polysaccharide.

According to a preferred embodiment, the solubilized polysaccharide is at a pH of between about 5.5 to about 10.

According to a further embodiment, crosslinking the solubilized polysaccharide with a methacrylate-containing molecule comprises mixing the solubilized polysaccharide with a methacrylate-containing molecule such as methacrylic anhydride, 2-hydroxyethyl methacrylate (HEMA), 2-hydroxypropyl methacrylate (HPMA), acrylamide (AAm), acrylic acid (AAc), N-isopropylacrylamide (NIPAm), methoxyl poly (ethylene glycol) (PEG) monoacrylate (mPEGMA or PEGMA), N,N′-methylenebis(acrylamide) (MBA), ethylene glycol diacrylate (EGDA) or PEG diacrylate (PEGDA), or glycidyl methacrylate for between about 1 hour to about 72 hours at a pH of between about 8 and 8.5.

According to a further embodiment, mixing the solubilized polysaccharide with the methacylate-containing molecule is carried out at a temperature of about 1° C. to about 15° C.

According to a further embodiment, crosslinking the solubilized polysaccharide comprises a) mixing the solubilized polysaccharide with the methacrylate-containing molecule such as methacrylic anhydride, 2-hydroxyethyl methacrylate (HEMA), 2-hydroxypropyl methacrylate (HPMA), acrylamide (AAm), acrylic acid (AAc), N-isopropylacrylamide (NIPAm), methoxyl poly (ethylene glycol) (PEG) monoacrylate (mPEGMA or PEGMA), N,N-methylenebis(acrylamide) (MBA), ethylene glycol diacrylate (EGDA) or PEG diacrylate (PEGDA), or glycidyl methacrylate and a catalyst such as pyridine and allowing it to react for about 0.5 hour to 3 hours at a temperature ranging from 40° C. to 60° C.; b) adding a highly concentrated alcohol to quench the reaction; and c) isolating the methacrylate-containing molecule by filtration, thereby creating the cross-linkable polysaccharide. In some cases, the catalyst could be triethylamine, N,N-diisopropylethylamine, N,N-diethylbenzylamine, or pyridine.

According to a further embodiment, the solubilized polysaccharide is subjected to filtration to remove residual water prior to mixing the solubilized polysaccharide with the methacrylate-containing molecule and the catalyst. According to a further embodiment, the solubilized polysaccharide is washed and mixed with a solvent to remove residual water prior to mixing the solubilized polysaccharide with the methacrylate-containing molecule and the catalyst.

According to a further embodiment, the water soluble polysaccharide is pectin, starch, amylose, amylopectin, glycogen, gum arabic, gum ghatti, gum karaya, pullulan, β-glucans, dextran, xanthan, alginate, gellan LA, levan or hyaluronic acid.

In another embodiment of the invention, a method of transforming a cross-linkable polysaccharide into a light cross-linkable polysaccharide is provided. The method involves providing a solution comprising a cross-linkable polysaccharide as described above and mixing the solution with about 0.1 to 0.4% w/w or % w/v of a photo-initiator in the dark, thereby making a solution comprising the light cross-linkable polysaccharide. According to a preferred embodiment, the method further comprises storing the solution comprising the light cross-linkable cross-linkable polysaccharide in the dark.

In another embodiment of the invention, a method of transforming a light cross-linkable polysaccharide into resin is provided. The method comprises providing a solution comprising a light cross-linkable polysaccharide as described above and exposing the solution to a wavelength of between about 320 nm to about 450 nm, preferably 405 nm, for between about 5 minutes to about 30 minutes.

In another embodiment of the invention, a method of preparing an intermediary methacrylated-amine molecule is provided. The method involves mixing a methacrylate-containing molecule and an amine-containing molecule to prepare a reaction mixture. The missing step is followed by adding a catalyst to the reaction mixture at a temperature ranging from 20° C.-50° C. to obtain the intermediary methacrylated-amine molecule.

In a non-limiting embodiment, the methacrylate-containing molecule is 2-hydroxyethyl methacrylate (HEMA), 2-hydroxypropyl methacrylate (HPMA), acrylamide (AAm), acrylic acid (AAc), N-isopropylacrylamide (NIPAm), methoxyl poly (ethylene glycol) (PEG) monoacrylate (mPEGMA or PEGMA), N,N-methylenebis(acrylamide) (MBA), ethylene glycol diacrylate (EGDA), PEG diacrylate (PEGDA), methacrylic anhydride, glycidyl methacrylate or any other suitable methacrylate-containing molecule.

In another non-limiting embodiment, the amine-containing molecule is ethylenediamine or any amine-containing molecule in the art. In some embodiments, the amine-containing molecule is ethylenediamine, diethylenetriamine 1,3-Diaminopropane, Putrescine, Cadaverine,1,2-Dimethylethylenediamine, Hexamethylenediamine or 1,1-Dimethylethylenediamine

In another non-limiting embodiment, the catalyst is triethylamine or any known catalyst in the art. In some cases, it could be triethylamine, N,N-diisopropylethylamine, N,N-diethylbenzylamine, or pyridine

In another non-limiting embodiment, the intermediary methacrylated-amine molecule is methacrylated ethylenediamine or any other methacrylated-amine molecule known in the art.

In another aspect of the invention, a method of transforming a polysaccharide into a cross-linkable polysaccharide is provided. The method involves preparing a mixture comprising a tosyl-polysaccharide and an intermediary methacrylated-amine molecule. Then the mixture is allowed to react for at least one hour at a temperature ranging from 70° C.-90° C., thereby obtaining the cross-linkable polysaccharide.

According to a further embodiment, the step of preparing the mixture comprises converting the polysaccharide to a tosyl-polysaccharide and simultaneously preparing the intermediary methacrylated-amine molecule according methods recited hereinbefore; and mixing the tosyl polysaccharide with the intermediary methacrylated-amine molecule to prepare the mixture.

According to a further embodiment, the method further involves precipitating the reaction mixture using alcohol to obtain a precipitate and filtering the precipitate using any filtration technique to obtain the cross-linkable polysaccharide.

According to a further embodiment, the method involves a step of converting the polysaccharide to the tosyl polysaccharide that involves mixing the polysaccharide in a solubilized form with triethylamine and a tosyl-containing molecule for about 24 hours at a temperature of up to about 30° C., thereby obtaining the tosyl-polysaccharide. In an alternate embodiment, the conversion step is carried out by mixing the polysaccharide in a solubilized form with a pyridine-ethanol solution and a tosyl-containing molecule at a low temperature for about 1 hour, thereby obtaining the tosyl-polysaccharide.

The invention is further directed to a cross-linkable polysaccharide obtained according to the method described above.

The invention is further directed to a light cross-linkable polysaccharide obtained according to the method described above.

The invention is further directed to a resin obtained according to the method described above.

Another aspect of the invention is directed to a use of the cross-linkable polysaccharide for preparing a light cross-linkable polysaccharide.

Another aspect of the invention is directed to a use of the light cross-linkable polysaccharide for preparing a resin.

In another embodiment of the invention, a method of preparing a protective coating is provided. The method involves mixing component A and component B. Component A is a cross-linkable polysaccharide or a light cross-linkable polysaccharide obtained according to the methods recited above. Component B is a base formulation with a diacrylate; a resin; and optionally a photo-initiator. The mixing step is followed by a step of curing the mixture by exposing it to UV light for a predetermined time, to obtain the protective coating.

According to a further embodiment, the mixing step involves dispersing component B in component A at high rpm with a stir bar.

According to a further embodiment, the mixing step is followed by a step of placing the mixture in a vacuum chamber to remove bubbles.

According to a further embodiment, the curing step is carried out after applying the mixture to a surface that needs to be coated.

According to a further embodiment, the mixture is exposed to UV light ranging from 390 nm to 415 nm for about 30 seconds to 5 minutes.

According to a further embodiment, the acrylate could be any known acrylate in the art such as isobornyl acrylate or 1,6-hexanediol diacrylate; the resin could be any known resin used in curing procedures such as aliphatic urethane acrylate or acrylated urethane, and the optional photo-initiator could be any known photo-initiator known in the art such as Darocur 1173.

In another embodiment of the invention, use of the protective coating prepared according to the above-recited method is provided. The protective coating can be used to prepare a nail polish coating, a varnish, a wood coating, a circuit board coating, a Teflon mold coating, an industrial coating, a plastic coating, a glass coating, a UV curable coatings, an automotive coating, a conformal coating, an optical coating, a coating for modulating hydrophobicity, or an all-purpose coating.

In another embodiment of the invention, use of the protective coating prepared according to the above-recited method is provided. The protective coating can be used for tissue engineering, tissue repair, bone tissue engineering, in-vivo or ex-vivo scaffold engineering applications, bio-ink applications, bone implants, dental treatment, dental implants, aesthetic applications, microfluidic devices and applications, microcarrier applications, medical treatment applications, wound treatment or stent applications.

Other and further aspects and advantages of the present invention will be better understood upon the reading of the illustrative embodiments about to be described or will be indicated in the appended claims, and various advantages not referred to herein will occur to one skilled in the art upon employment of the invention in practice.

### Experimental Data

A novel method of transforming a polysaccharide into a cross-linkable molecule will be described hereinafter. Although the invention is described in terms of specific illustrative embodiments, it is to be understood that the embodiments described herein are by way of example only and that the scope of the invention is not intended to be limited thereby.

A novel method to transform naturally occurring, neutral polysaccharides (e.g., cellulose nanofibrils (CNFs)) into a cross-linkable molecule has been developed and will be described herein. The polysaccharide is first converted into an amino-polysaccharide derivative by replacing hydroxyl groups with amine groups which are then linked to methacrylate groups, thereby conferring cross-linking abilities to the methacrylated-linked polysaccharide. The simplified two-step method will be described in detail. The method being applied to CNFs for transforming CNFs into biocompatible water-soluble bio-ink which can be used for DLP UV 3D printing will be described in the examples. Detailed characteristics of this highly customizable form of cellulose derivative will also be described. Moreover, additional functional groups can be added to the cellulose along with UV curing capability (including but not limited to proteins, growth factors, etc.). Advantageously, the method can be applied to various neutral polysaccharides.

Also described is a novel method of transforming a water-soluble polysaccharide into a cross-linkable molecule by grafting methacrylate groups directly to a water-soluble polysaccharide (e.g., pectin) using a semi-heterogeneous reaction that tunes the polysaccharide to react under a specific state of protonation in high concentration reactants. Advantageously, the semi-heterogeneous reaction allows for low post-processing steps, thus reducing the energy input and environmental impact, as the method uses water as solvent.

The methods described herein provide a simplified approach to functionalizing polysaccharides (e.g., cellulose, pectin, etc.) to create light-cross-linkable molecules. The methods use the strategy of inserting nucleophilic groups or promoting the nucleophilicity of reactive groups as intermediaries to methacrylation. This leads to a significant improvement in the process of methacrylation and allows for the creation of covalently crosslinked carbohydrate-based materials.

The method described has been developed to produce a cellulose-based, water-soluble, UV curable resin by substituting one or more —OH group(s) from the glucose sub-units primarily at carbon-6 (C6, other groups at different carbon positions can be substituted) in the cellulose with ethylenediamine. To this (or those) linker(s), methacrylate is attached, which confers cross-linking abilities to the molecule.

The method includes dissolving a polysaccharide in a solvent, preferably a neutral polysaccharide in a polar solvent. The polar solvent is preferably aprotic. A neutral polysaccharide may for example be dissolved in dimethylacetamide (DMAc) together with an inorganic salt such as lithium chloride (LiCl) or lithium bromide (LiBr). Other suitable solvents include polar aprotic solvents such as dimethyl sulfoxide (DMSO) and dimethylformamide (DMF); ionic liquids such as 1-butyl-3-methylimidazolium chloride (BMIMCl) and 1-allyl3-methylimidazolium chloride (AMIMCl); and alkali solvents such as a solution containing NaOH and urea.

For example, lyophilized CNFs may be dissolved in a solution comprising CNFs, dimethylacetamide, and lithium chloride in a 1:50:4 ratio respectively. The solution may be heated to accelerate dissolution to a temperature of about 100° C. until lithium chloride is fully dissolved and the solution is homogenous. The solubilization of the neutral polysaccharide may be performed at room temperature up to about 200° C., depending on the humidity levels of the polysaccharide. For a completely dried polysaccharide, lower temperatures would be sufficient. Alternatively, the neutral polysaccharide may be heated to a boiling temperature prior to adding the LiCl. The resulting solution is a mixture of DMAc, LiCl and CNFs. Once the CNFs are dissolved, the nucleophilicity of the CNFs is enhanced either by inserting nucleophilic groups or by promoting the nucleophilicity of reactive groups within CNFs as intermediates between CNFs and the cross-linkable molecules. For example, tosylation and subsequent amination of CNF may be performed as previously described (7, 8).

The solution is then cooled to room temperature once the CNFs and lithium chloride are fully dissolved. p-toluenesulfonyl chloride is then added to the mixture to a final concentration of about 5-20 mM and triethylamine (TEA) to a final concentration of about 1-5 mM and allowed to react with reactive groups within CNFs such as hydroxyl groups in order to generate tosylate CNFs. The tosylation reaction is allowed to react for about 24 hours at room temperature while stirring. Alternatively, the reaction may be cooled to about 0° C. and allowed to reach up to about 48 hours or the reaction may be heated and allowed to react for at least an hour.

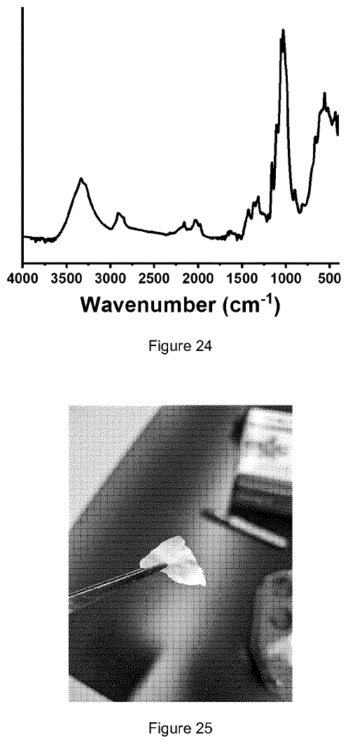

Alternatively, the tosylation reaction can be carried out using a pyridine-ethanol solution. In an exemplary embodiment, the method involves transferring 3% CNF slurry (in water) to a centrifuge tube followed by centrifugation at 4000 rpm for 10 minutes. The supernatant is removed and approximately 25 mL of 99% ethanol is added to the centrifuge tube. The centrifuge tube is agitated to resuspend the cellulose and these steps are repeated 3 times to exchange the solvents. p-Toluenesulfonyl chloride (TsCl) is dissolved in 50 mL of ethanol. In a separate container, a pyridine-ethanol solution is prepared by adding pyridine to ethanol. While stirring the cellulose slurry with ethanol, the pyridine-ethanol solution is gradually added to facilitate the reaction process. Then, the dissolved TsCl solution is added to the cellulose slurry while stirring continuously. After complete addition of the TsCl solution, stirring of the reaction mixture is continued at a low temperature, between 0-5° C., for 1 hour. The reaction mixture is then poured onto a filter and vacuum filtration is initiated, and the filtrated solids are washed on the filter with additional 99% ethanol. After washing, the tosylated CNF (tosylCell) is allowed to dry with vacuum drying. The tosyl can be dissolved in ethanol (EtOH) and a solvent exchange can be carried out to ethanol for the CNF slurry, ensuring compatibility and better modification of the CNF. This protocol allows for surface modification of the CNF. The FTIR spectrum of CNF to tosylCell is provided in FIG. 24 where the procedure involves solvent exchange of the slurry with 99% Ethanol. The tosylCell obtained after solvent exchange with the CNF slurry and reaction is shown in FIG. 25.

It is to be understood that other time, mixing, drying and temperature parameters may be implemented and adjusted according to specific needs while remaining within the teachings of the present invention. It is further to be understood that other reactive groups may be allowed to react according to the selected polysaccharide while remaining within the teachings of the present invention. Examples of alternative reactive groups include methoxyl; methyl; ether; ester; carbonyl and carboxylic acid which may be targeted depending on their availability on the selected polysaccharide.

The tosylate CNFs are then converted into aminated CNFs by amination of CNFs, which enhances the nucleophilicity of reactive groups within CNFs prior to crosslinking with methacrylate. The tosylate CNFs are extracted from the dimethylacetamide solution by precipitation with a polar solvent. For example, tosylate CNFs may be precipitated with methanol, ethanol, propanol, acetone, toluene, xylene, water or any other suitable polar solvent. The precipitate containing tosylated CNFs is then dissolved in dimethyl sulfoxide (DMSO) or any other suitable solvent such as DMAc/LiCl, N-methylmorpholine N-oxide, ionic liquids or urea/NaOH before adding ethylenediamine (EDA) to the mixture. Other amine-containing molecules may be used while remaining within the scope of the present invention. For example, 1,4-butanediamine, pentane-1,5-diamine, N-(3-Aminopropyl)butane-1,4-diamine, poly(ethylene) amines or any other suitable amine-containing molecule may be used. The solution containing tosylate CNFs and EDA is then heated to about 100° C. for about for 24 hours to allow amination of the tosylate CNFs and generate aminated CNFs. The amination reaction may also be heated up to about 150° C. The aminated CNFs are then extracted by precipitation with a polar solvent such as ethanol, acetone, toluene, xylene, water or any other suitable polar solvent. The precipitate containing the aminated CNFs is then dissolved in an aqueous solution such as water, Phosphate-Buffer Saline (PBS) or any other suitable buffer solution, irrigation saline solution at about 9%, cell culture media or any other suitable solvent according to a specific need, for example, according to a specific downstream application. A methacrylate-containing molecule such as glycidyl methacrylate (GMA) is then added to the mixture containing aminated CNFs and incubated for about 24 hours while stirring to allow the methacrylate molecules to attach to the amine group of the CNFs via epoxy ring-opening. Other suitable methacrylate-containing molecules include, but are not limited to methacrylic anhydride, methacrylic acid, hydroxyethyl methacrylate, methyl methacrylate, ethyl methacrylate and benzyl methacrylate. The mixture may be incubated from about 1 hour to about 48 hours depending on the reaction temperature and other selected reaction parameters. Other selected parameters may include pH, temperature, desired group substitution as well as the nature of the methacrylate-containing molecule. The methacrylated CNFs are then extracted by precipitation with a polar solvent such as ethanol, acetone, toluene, xylene, water or any other suitable polar solvent. Alternatively, EDC/NHS can be added to activate the reaction. The resulting solid precipitate comprising the cross-linkable molecule, methacrylated CNF, is then dissolved in water and kept at 4° C. for downstream use.

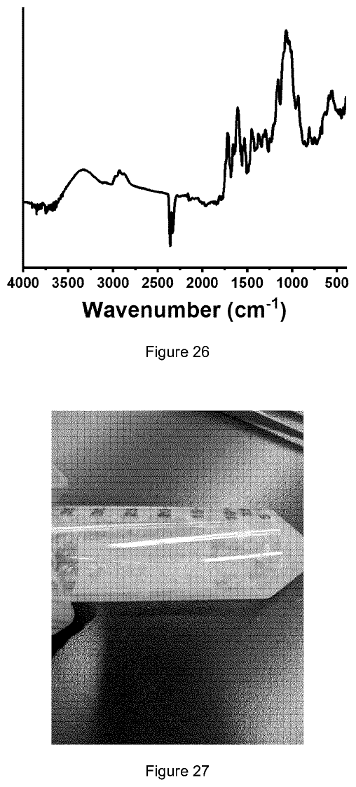

Alternatively, the aminated CNF's or amino cellulose (aminoCell) can be converted to methacrylated CNF using various other protocols. These protocols are time efficient and maintain the quality of the end product (i.e. mCNF). For instance, in a beaker, aminated CNF or aminoCell can be dissolved in water. The temperature is then set to 50° C. Next, methacrylic anhydride is added to the mixture, followed by vigorous stirring for 1 hour. The resulting solution is then placed in excess 99% ethanol and subjected to vacuum filtration. The obtained solid is allowed to dry and subsequently transferred into a 3500 mwco (molecular weight cutoff) dialysis tube for 3 days, against water. The FTIR spectrum of preparation of methacrylated CNF (mCNF) using methacrylic anhydride i.e. the reaction of aminoCell with methacrylic anhydride is provided in FIG. 26. The dry solids in the dialysis tube is shown in FIG. 27.

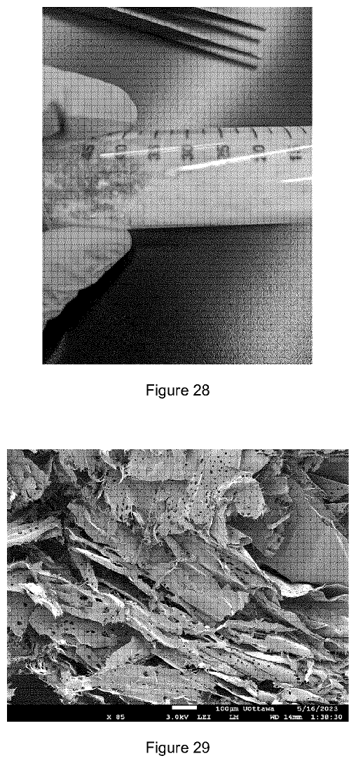

Alternatively, the conversion process can be achieved using glycidyl methacrylate. In a beaker, aminoCell or aminated CNF can be dissolved in water. The temperature is set to 50° C. Next, glycidyl methacrylate (GMA) is slowly added, dropwise, over the course of 10 min. The mixture is then stirred for 1 hour, then additional water is added. The resulting solution is then placed in excess 99% ethanol and subjected to vacuum filtration. The reaction mixture is cooled down to room temperature, removed from the heating setup and transferred into a 3500 mwco (molecular weight cutoff) dialysis tube for 3 days, against water. The dry solids obtained using this method in the dialysis tube are shown in FIG. 28. The scanning electron microscope image of methacrylated CNF obtained using GMA i.e via reaction of aminoCell and glycidyl methacrylate is shown in FIG. 29.

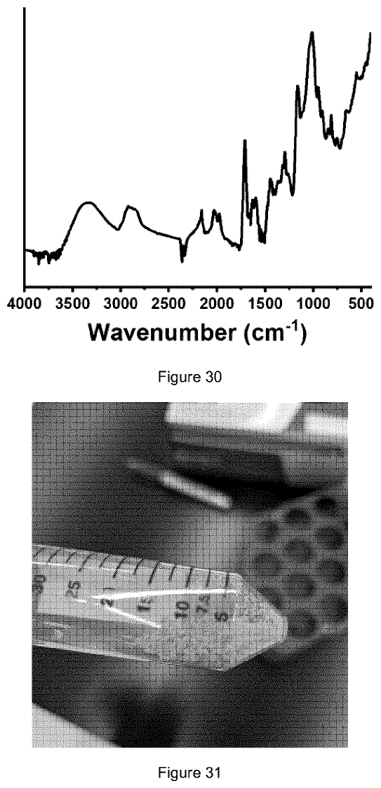

Alternatively, the conversion process can be achieved using glycidyl methacrylate via the following protocol. The amino cellulose or aminated CNF is added to a round bottom flask, at room temperature. This is followed by adding DMSO to the flask and mixed with a stir bar until dissolution. Glycidyl methacrylate is then added and the temperature is raised to approximately 70° C. While still at room temperature, triethylamine (TEA) is added. Once the temperature reaches 70° C., the mixture is allowed to react for 1 hour. After 1 hour, the reaction mixture is allowed to cool down to room temperature, and removed from the heating setup. The solution is then precipitated by adding the reaction mixture to excess ethanol 99%. The precipitate is then collected by filtration and washed with ethanol to remove impurities. The purified product is dried in a vacuum desiccator until it is fully dry. It is to be noted that the temperature was increased before adding the reagents. The FTIR spectrum of mCNF i.e. products from the reaction of aminoCell with glycidyl methacrylate and triethylamine is provided in FIG. 30. The dried mCNF obtained following reaction of GMA with TEA catalyst is shown in FIG. 31.

The chemical structure of cellulose, methacrylated amino cellulose and the different reaction intermediates are depicted below.

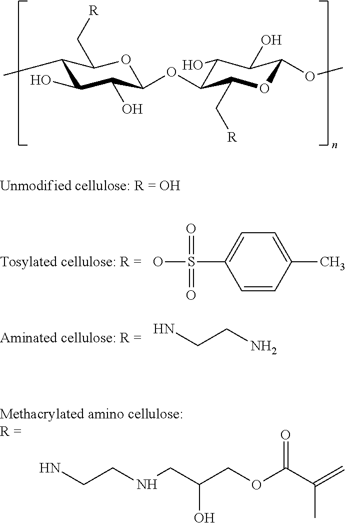

Another aspect of the invention is a novel method of transforming water-soluble polysaccharides such as glucans, guaran, glycogen, starch (amylose and amylopectin), pullulan, xylans and anionic polysaccharides such as pectin, rhamnogalacturonans, alginate, gum arabic, gum acacia, gum ghatti into cross-linkable molecules selectively functionalizing photosensitive linkers on carbon-2. The method includes dissolving a water-soluble polysaccharide in water to a concentration of between about 0.3% to about 4% w/v and adjusting the pH between about 8 to about 8.5 by adding a suitable base, for example, sodium hydroxide. A methacrylate-containing molecule such as methacrylic anhydride is then added dropwise to the mixture to a final concentration of about 10% v/v while maintaining the pH between about 8 to about 8.5 using sodium hydroxide. The reactive solution is incubated while stirring at room temperature for about 10 min and then incubated for about 24 hours at a temperature of about 5° C. to 10° C. while stirring. The solution is then precipitated in cold ethanol, methanol or acetone and the precipitate is dialyzed against deionized water and then lyophilized for storage. The chemical reaction of pectin with methacrylic is depicted below.

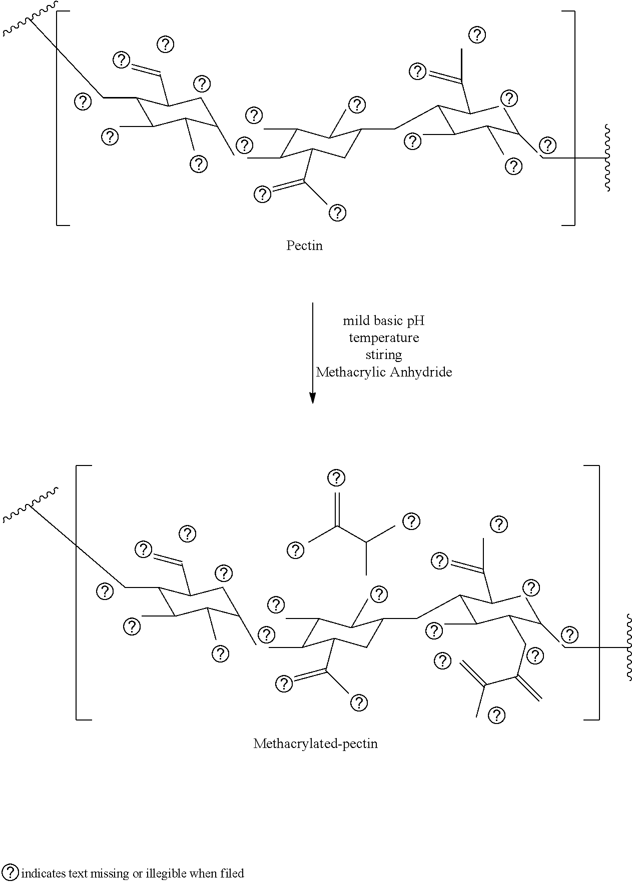

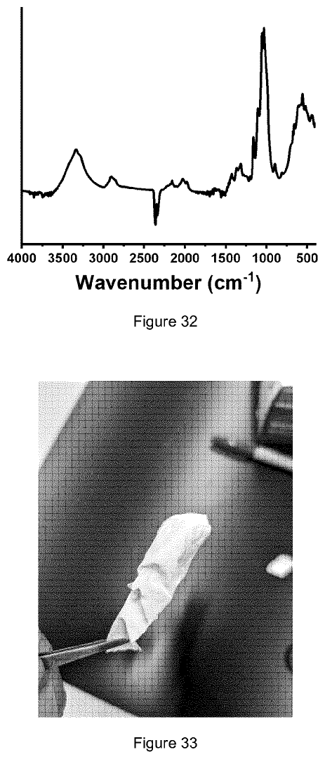

In an alternate embodiment, a novel method of transforming a water-soluble polysaccharide (CNF) to methacrylate CNF (mCNF) is provided. The process involves a solvent exchange. Firstly, set up a filtration system using a vacuum filter, to separate the CNF slurry (in water) from the liquid phase. Following this, the cellulose solids are washed with DMSO to remove any residual water and to replace it with DMSO. The excess DMSO is allowed to drain. Next, the CNF slurry is transferred into a round bottom flask and DMSO is added, ensuring thorough mixing to disperse the cellulose. This is followed by adding an equal molar amount of methacrylic anhydride and pyridine. The reaction mixture is then stirred to ensure proper mixing. The temperature of the reaction vessel is maintained at 50° C. and the mixture is stirred for 1 hour. The reaction is quenched by adding the solution to 99% ethanol. The mCNF is then isolated by vacuum filtration and the modified cellulose is dried under vacuum. The FTIR spectrum of mCNF i.e products from the reaction of CNF in suspension in DMSO with methacrylic anhydride and pyridine is provided in FIG. 32. The isolated mCNF, following reaction of CNF slurry (in DMSO) with methacrylic anhydride and pyridine as a catalyst is shown in FIG. 33.

Another aspect of the invention is a novel method of transforming a cross-linkable polysaccharide into a light cross-linkable polysaccharide. The method comprises providing a solution comprising a cross-linkable polysaccharide, as described above for example, and mixing the solution with about 0.1 to 0.4% w/w or % w/v of a photo-initiator. The photoinitiator bis(acyl)phosphane oxi lithium phenyl-2,4,6-trimethyl benzoyl phosphinate (LAP) is an example of a suitable photoinitiator which may be added to a methacrylate CNF or a methacrylate pectin solution. The resulting solution comprising the light cross-linkable polysaccharide is then kept in the dark for downstream uses.

Another aspect of the invention is a novel method of transforming a light cross-linkable polysaccharide into resin. The method comprises exposing the solution comprising light cross-linkable polysaccharide to a wavelength of about 405 nm for about two intervals of about 5 minutes each. The solution may alternatively be exposed for an uninterrupted period of up to 10 minutes or any number of intervals ranging from 2 to about 100 from about 10 seconds to about 10 minutes.

In another aspect of the invention, the inventors have developed a faster and more efficient technique to obtain methacrylate CNF (mCNF). In this technique, mCNF is obtained from tosylCNF and involves synthesizing an intermediary. There's a significant time reduction as this technique allows generating mCNF in a matter of hours. In some cases, the time reduction is almost about 72 hours.

### Step 1: Preparation of Methacrylated Ethylenediamine (Intermediary) with Triethylamine Catalyst

In round bottom flask, an equimolar amount of methacrylic anhydride and ethylenediamine is mixed. Then, triethylamine (TEA) catalyst is added to the mixture. The mixtures is stirred continuously at room temperature or at 50° C. for 2 hours. Once the reaction is complete, the resulting methacrylated ethylenediamine (Intermediary) product is now ready for use in the subsequent step of the process.

It is to be noted that the above reaction is highly exothermic after it passes +−70° C. Therefore, a cooling bath could be used to slow down the reaction and to allow the reactants to react over longer period of time. This can also help in avoiding cross-linking or undesired side-products.

### Step 2: Addition of Methacrylated Ethylenediamine to Tosylated Cellulose

Tosylated cellulose (tosylCell) can be prepared simultaneously as explained earlier and dissolved in DMSO at 125° C. In a separate container, a solution of the methacrylated ethylenediamine (Intermediary) from Step 1 by diluting it with DMSO. The intermediary solution is then added to the tosylCell solution, while ensuring good mixing and uniform distribution. The mixtures are allowed to react for at least 1 h at 80° C. After the reaction, the solution is precipitated into 99% ethanol. The precipitated modified cellulose is collected by decantation in the non-solvent. Finally, the modified cellulose is thoroughly dried under vacuum. The resulting product mCNF is a clear, plastic-like solid. It is understood, that various modifications, of the above process are encompassed in this invention. The temperature limits can be varied, and the mixing, vacuum, filtration and other steps can be replaced with other techniques known in the art. Other suitable solvents could be used to replace DMSO.

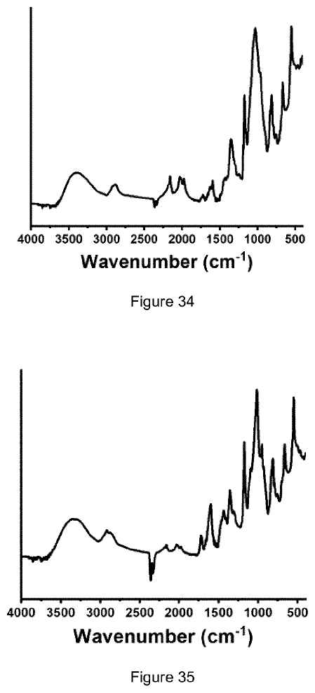

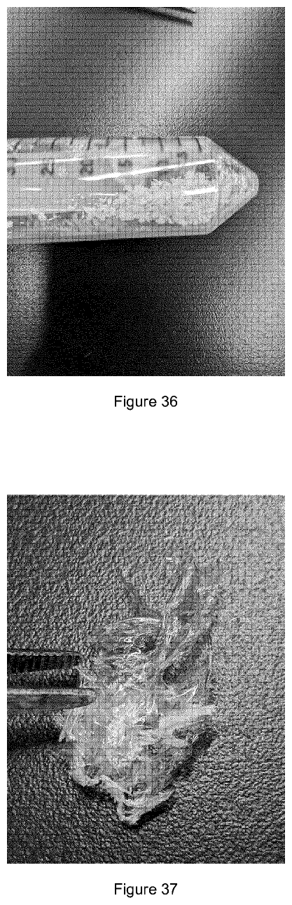

The FTIR spectrum of tosylCell to mCNF (via intermediary) where the intermediary reacted at room temperature is shown in FIG. 34. The FTIR spectrum of tosylCell to mCNF (via intermediary) where the intermediary reacted at 50° C. is shown in FIG. 35. FIG. 36 shows the mCNF (via intermediary) generated when the intermediary is reacted at room temperature. FIG. 37 shows the mCNF (via intermediary) generated when the intermediary is reacted at 50° C. s

A non-limiting example of the chemical reactions involved in Step 1 and Step 2 of the process is shown below.

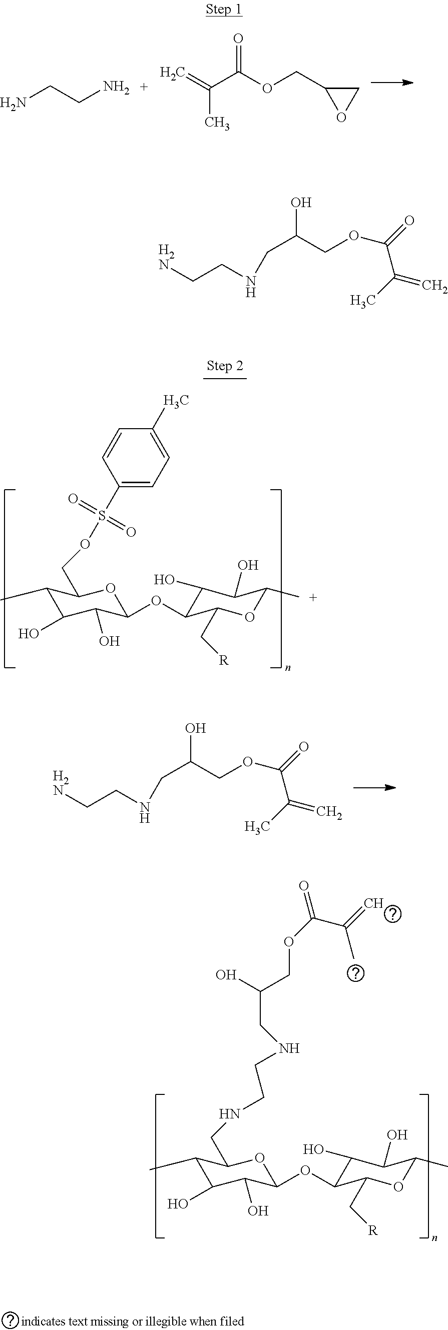

In each step described above, the temperature, mixing, concentration of the reactive species and solvents, pH, time of reaction, pressure and other physical parameters should be monitored continuously as variations may have an impact on group substitution, oxidation, the coloration of the material, cross-linking capability under UV, and self-crosslinking in solution (e.g. too high concentration causes the solution to self-crosslink).

### Example 1—Cross-Linkable Cellulose Derivative to Create Hydrogels and Aerogels for Bone Tissue Engineering

The naturally occurring lignocellulosic architecture of plants has been utilized for biological applications (2, 9-13). For instance, asparagus scaffolds were demonstrated to be a successful vector for spinal cord regenerations in vivo (14). Moreover, apple hypanthium tissue is a biocompatible, implantable scaffold that can be used for soft tissue and bone tissue engineering (2, 9-13). Other types of plants were also studied in a bioengineering context (2, 9-14). While keeping the native structure of the plant is advantageous in replicating targeted tissues and organs (2, 9-13), it has physical limitations. For instance, the scaling of the implant is limited by the original core material size and the microstructure can be inhomogeneous, with unexpected defects and shapes. Moreover, plant-derived cellulose scaffolds have a relatively low Young's modulus, limiting their use for certain BTE applications, due to possible stress shielding and lack of load-bearing capabilities.

An example of a method of transforming cellulose into cross-linkable cellulose will now be described. Lyophilised CNFs were placed in a 250 ml round bottom flask with dimethylacetamide in a 1:50 ratio, respectively. The temperature of the mixture was brought to 115° C. for 15 min. Lithium Chloride (LiCl) was added in a 4:1 ratio with respect CNFs, under vacuum at 100° C. until LiCl was fully dissolved. After dissolution, the mixture was slowly cooled down to room temperature. Tosylation was performed in a similar fashion as previously demonstrated (8). Briefly, 2.61 mM of triethylamine (TEA) and 13.11 mM of Tosyl were added. The solution was allowed to react for 24 h by stirring at room temperature. The resulting solution was precipitated in 99% ethanol and filtered under vacuum to remove the solvent. The solid phase was dissolved in DMSO at a concentration of 1 g/mL. Once dissolved, ethylenediamine (EDA) was added and the mixture was stirred for 24 h. The resulting solution was precipitated in cold 99% ethanol and filtered under vacuum to remove the solvent. The solid phase was dissolved in dH₂O at a 1:10 ratio, respectively. 18.91 mM of glycidyl methacrylate (GMA) was added and the solution was stirred at 75° C. for 24 h before precipitation in cold 99% ethanol and centrifugation. The resulting solid (mCNF) was dissolved in water at different concentrations. Alternatively, 11.27 mM of EDC and 2.61 mM of NHS were added to the mixture. Characteristics of CNF, mCNF and the reaction intermediates will now be described.

### Fourier Transform Infrared Spectra

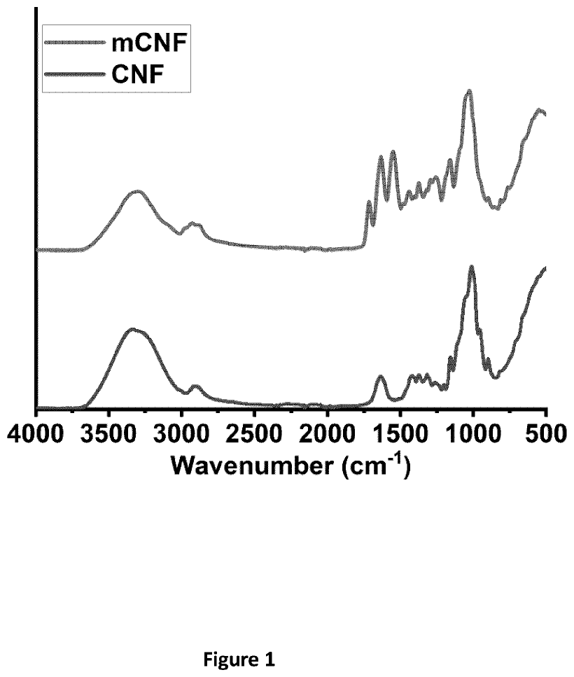

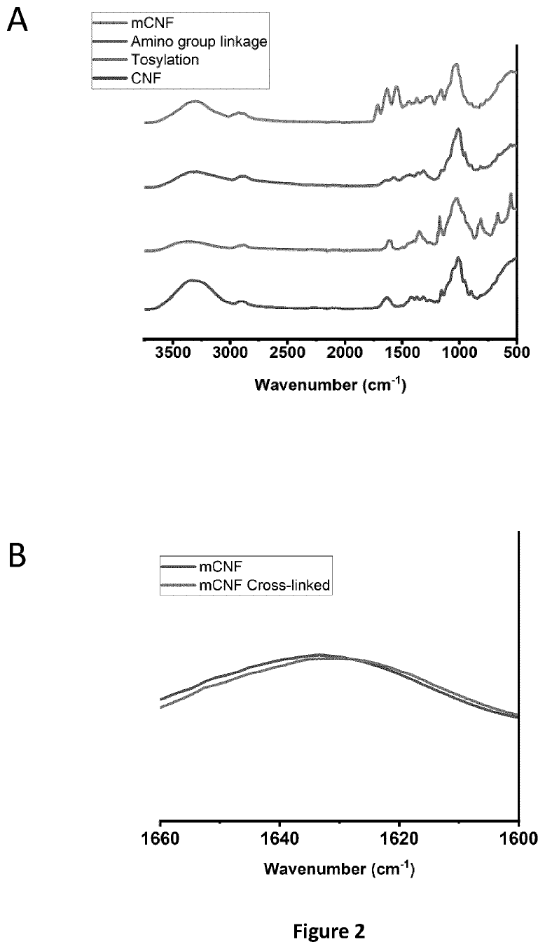

Samples at different reaction stages were frozen at −80° C. overnight and lyophilized for 24 h before FTIR analysis. Spectra were recorded with a Nicolet 6700 AT-FTIR from 4000 to 500 cm-1. FIG. 1 show representative spectra of methacrylated cellulose nanofibrils compared to pristine cellulose nanofibrils whereas FIG. 2 shows a Fourier transform infrared spectra of cellulose nanofibrils, methacrylated cellulose, intermediary steps and crosslinked methacrylated cellulose reacted with GMA. Peaks at 3300 cm⁻¹ and 2915 cm⁻¹ are visible for both mCNF resin and pristine CNF, showing characteristic alcohol group and alkane stretching vibrations, respectively. At 1012 cm⁻¹, characteristic C—C stretching vibrations of pyranose rings of cellulose nanofibrils are shifted towards 1025 cm⁻¹ for mCNF. Alcohol group bending is also visible for both curves at around 1632 cm⁻¹. New absorption peaks are visible for the mCNF curve at 1714 cm⁻¹ and 1550 cm⁻¹.

### Nuclear Magnetic Resonance Spectroscopy

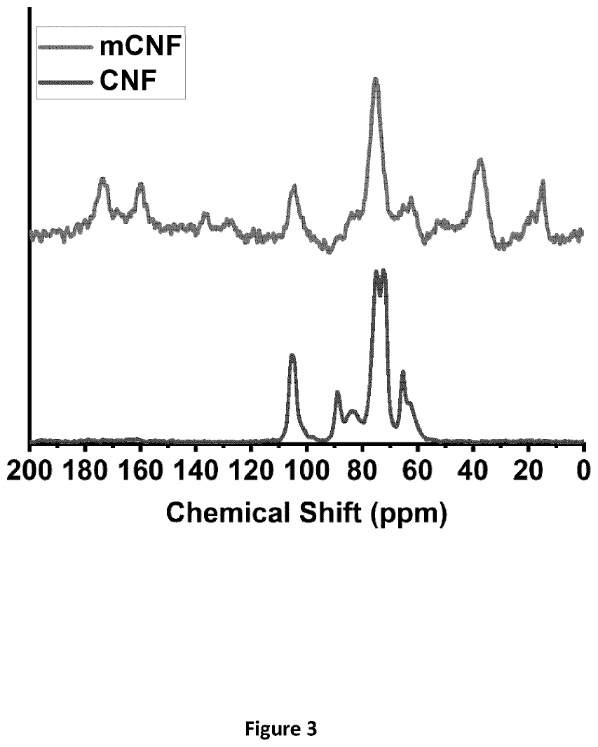

Samples at different reaction stages were frozen at −80° C. overnight and lyophilized for 24 h before NMR analysis. Solid state Carbon-13 Magic angle spinning NMR spectra were recorded with a Bruker Avance III 200. Spectra were collected at a 200 MHz frequency. FIG. 3 shows a ¹³C solid state NMR spectra of UV cross-linked methacrylated cellulose nanofibrils compared to pristine cellulose nanofibrils. Spectra of pristine CNF is showing characteristic carbon chemical shifts of cellulose at 105.2 ppm; 88.9 ppm; 75.0 ppm, 72.5 ppm and 65.3 ppm; 62.7 ppm, representing C-1; C-4; C-2, C-3, C-4; C-6 on the cellulose chain. At 173.33 ppm and 160.12 ppm of the mCNF spectra, peaks of methacrylate carboxylic acid and carbon-oxygen double bonds are visible, respectively. Carbon-carbon double bond doublet is visible in the mCNF NMR spectra at 136.61 ppm and 127.75 ppm. Additionally, the CH₂ signal from the attached ethylenediamine is visible at 37.17 ppm. Finally, the methyl signal from the methacrylate group is visible at 14.78 ppm. FTIR and NRM data demonstrate the chemical modification of ethylenediamine-modified CNF with GMA.

Transforming Cross-Linkable Cellulose into Light Cross-Linkable Cellulose

To transform the cross-linkable cellulose into a light cross-linkable molecule, a photoinitiator (LAP) was added to a solution comprising 0.2% w/v. The resulting light cross-linkable cellulose solution was kept at room temperature in the dark until use.

Transforming Light Cross-Linkable Cellulose into Hydrogels and Aerogels

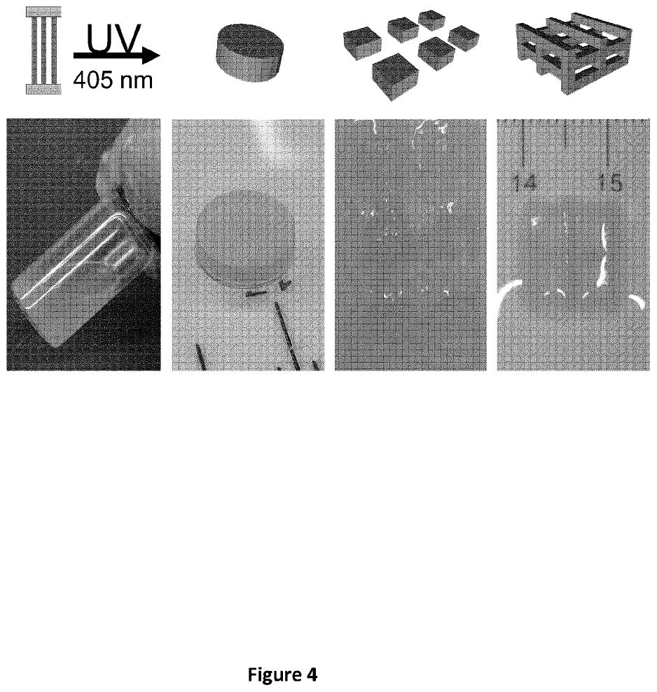

Transforming the light cross-linkable cellulose into hydrogels was performed by exposing mCNF resin (0.5 g/mL) with UV light (FIG. 4). Disk-shaped hydrogels were constructed by pipetting 50 μL of the resin into a 6 mm diameter Teflon mold. The constructs were exposed for 10 min with 405 nm light. Three-dimensional cubes and haystack hydrogels were printed using the LumenX UV DLP printer by pipetting 1 mL of mCNF resin (0.5 g/mL) in the printing vat. Each layer was exposed for 10 s with 405 nm light.

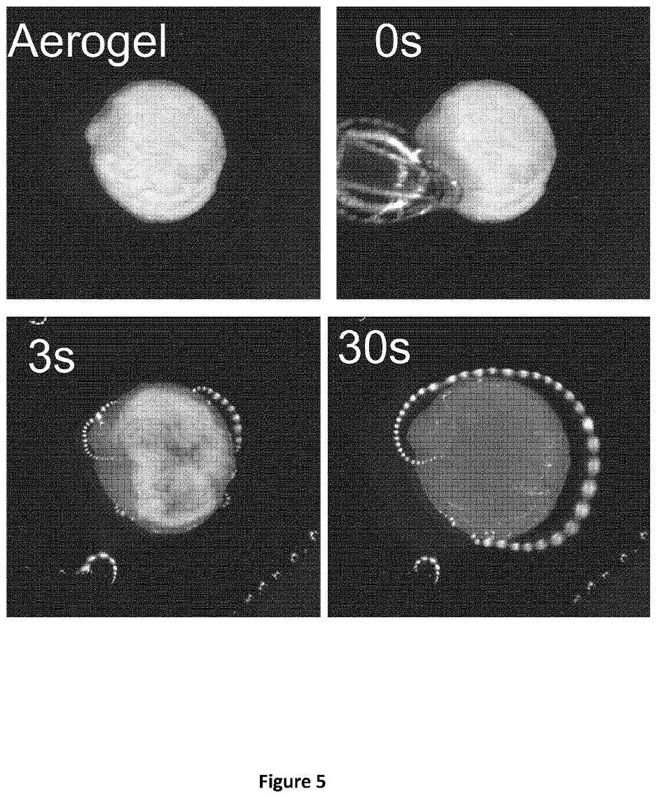

Aerogels were constructed by lyophilizing the hydrogel constructs. Disk-shape hydrogels were frozen at −80 C for 24 h. Frozen hydrogels were placed in a dry freezer for 24 hours, resulting in a porous aerogel. Aerogels were rehydrated by pipetting 10 μL of cell culture media on the aerogels until reabsorption (FIG. 5). Over a period of 30 seconds, the culture media completely infiltrated and rehydrated the aerogels. Characteristics and exemplary uses of the resin will now be described.

### Surface Characterization

Scanning Electronic microscopy (SEM) was performed to observe the surface features of Hydrogels and Aerogels. Hydrogels were serially dried in increasing concentration of ethanol, from 70% to 99%. Hydrogels in ethanol were dried in a critical point dryer following manufacturer's protocol. Hydrogels and aerogels were coated with a 9 mm gold layer before SEM imaging. Images were acquired with a JEOL JSM-7500F FESEM scanning electron microscope at 85× magnification (N=3 for hydrogels; N=3 for aerogels). SEM image analysis was used to evaluate the surface of the hydrogels and to observe the pore formation of the aerogels (FIG. 6). Hydrogels are displaying an opaque, uneven surface. Freezing at −80° C. and lyophilization of the hydrogels created a distinct porous structure that resulted in aerogels, with the removal of water from the constructs.

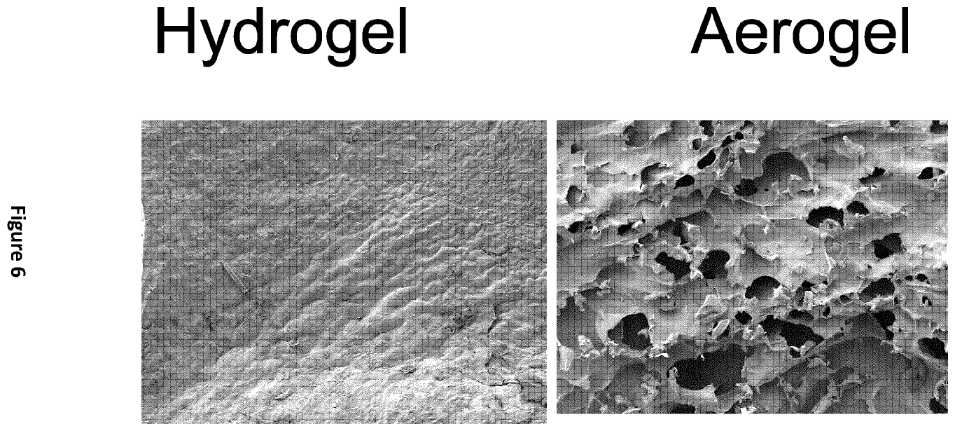

Porosity measurements of aerogels were performed using histological sectioning and image analysis. Rehydrated aerogels were fixed with 10% formalin for 30 min and placed in 70% ethanol. Paraffin embedding and staining of the sample sectioning (4 μm thick) were performed by the PALM Histology Core Facility at the University of Ottawa. Slides were stained with Masson Trichrome and images were acquired with a Zeiss AXIOVERT 40 CFL microscope at a 40× magnification. A 555 by 555 μm2 region of interest (ROI) was randomly selected on samples and the images were threshold using ImageJ software to highlight pores in the samples. The pore size was registered as the major axis (N=6 samples; 3 ROI per sample). The porosity of the aerogels was quantified using histological sections and staining (FIG. 7). Results showed an average pore size of 20.5±0.1 μm, with a distribution of pore size between 6.5 μm and 228.8 μm. We observed a median pore size of 16 μm.

Mechanical Characterization of mCNF, Hydrogel and Aerogel

Rheometric analysis was performed to evaluate crosslinking kinetics, storage modulus and yield point using a rheometer (Anton Paar GmbH). Oscillating shear stress was applied to the mCNF solutions to evaluate crosslinking kinetics and storage modulus plateau after periodic UV illumination. Briefly, 60 μl of mCNF at different concentrations (0.1 g/mL; 0.5 g/mL) was pipetted on the glass surface of the rheometer, equipped with parallel plate configuration. Samples were kept hydrated by pipetting the outer part of the apparatus with a 0.2% LAP solution in dH₂O. Samples were exposed to 405 nm light for 1 min and oscillating shear stress was applied at fixed amplitude (0.25% in 30 sec) for 210 s. Then, the samples were again exposed to 405 UV light for 1 min and oscillating sheer-stress was re-applied with the same conditions. The illumination/sheer-stress cycle was repeated 5 times. After the first 405 nm exposure, the storage modulus of the sample at 0.5 g/mL (31.5±0.4 kPa) was significantly higher than at 0.1 g/mL (0.272±0.002 kPa) (FIG. 8). This significant difference remained for all the exposures. We observed a significant increase (49-fold) in the final storage modulus of the 0.5 g/mL sample (71.5±0.4 kPa) compared to the 0.1 g/mL sample (1.449±0.002 kPa).

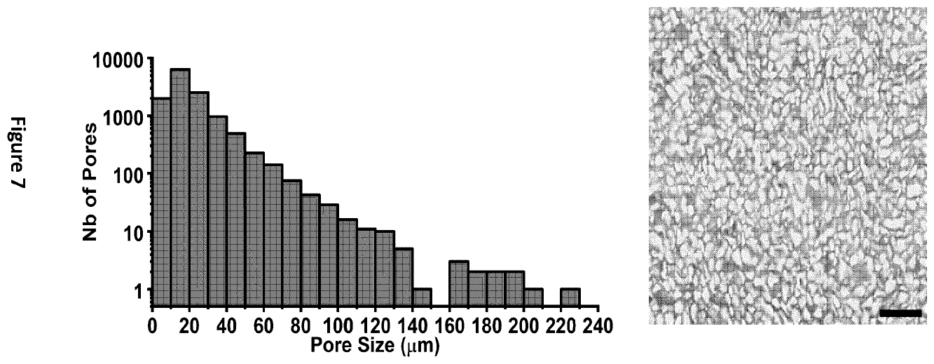

Amplitude sweep was performed in similar fashion, with the sample incrementally strained until a breaking point is observed, corresponding to the yield point. Briefly, 60 μl of mCNF resin at different concentrations (0.1 g/mL; 0.5 g/mL) was pipetted on the glass surface and was illuminated with 405 nm light for 5 minutes. Samples were sheered at a fixed frequency (1 Hz) with increasing sheer amplitude until a breakpoint was observed. Results (FIG. 9) revealed a plateau in the storage modulus for both concentrations of mCNF, below 5% strain. At 0.1 g/mL concentration of mCNF, we observed a yield point at 25% strain. Alternatively, the increase of concentration resulted in a lower yield point: a brittle fracturing occurred at 6% strain for the 0.5 g/mL mCNF.

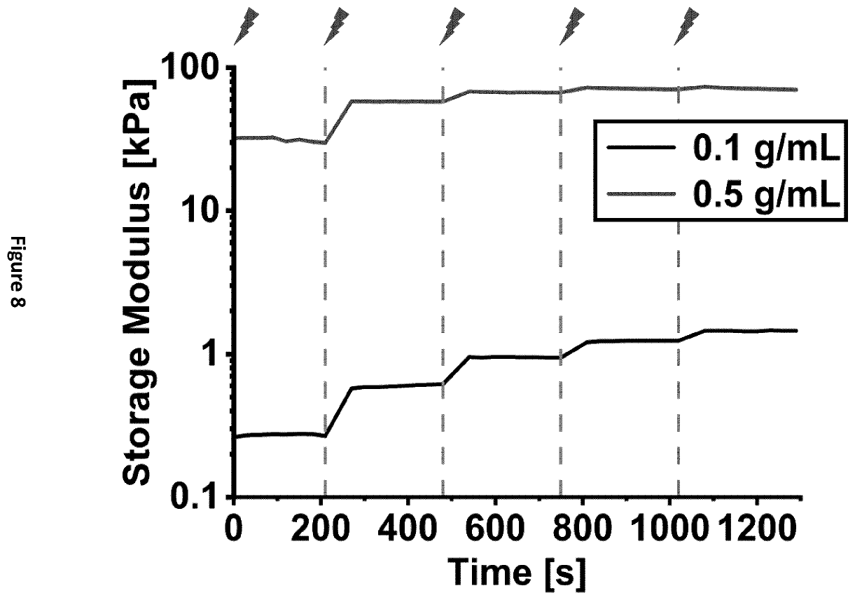

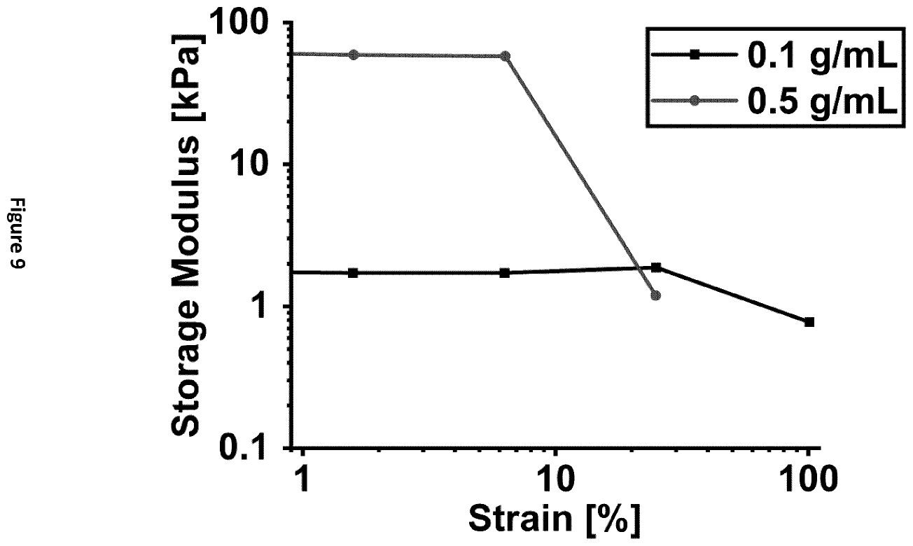

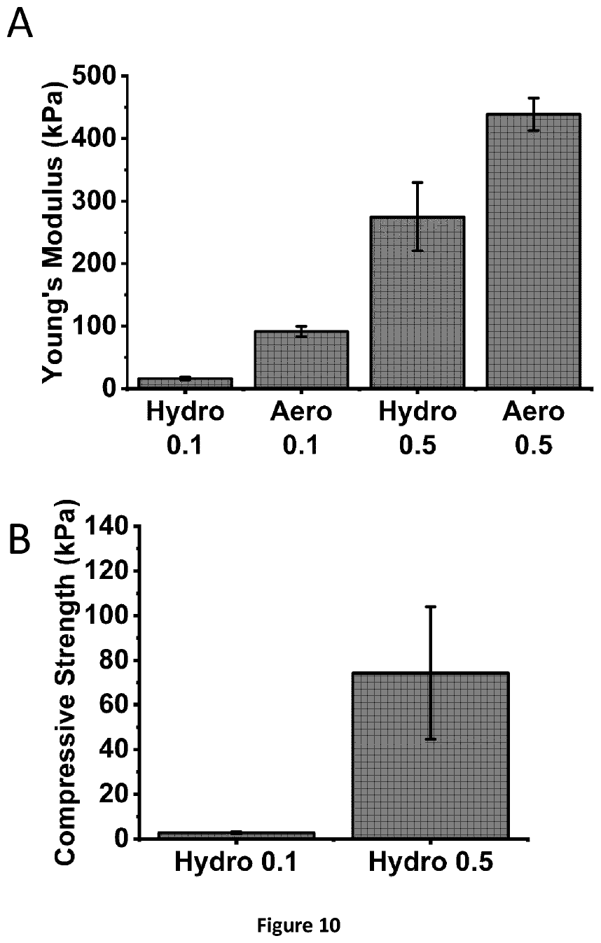

Young's moduli (YM) of both hydrogels and aerogels, at different concentrations (0.1 g/mL; 0.5 g/mL) were measured in a uniaxial compression experiment. Samples (Greater ≥3 for each experimental condition) were compressed at 1%/sec and the resulting force-displacement was recorded using a CellScale UniVert (CellScale, Waterloo, ON). YM was obtained by fitting the linear portion of the stress-strain curve. Compressive strength was obtained by compressing the sample until brittle fracturing occurred. Compressive strength was recorded as peak force before failure. Results showed a significant increase in YM of the hydrogels with an increasing concentration of mCNF (274.88±54.8 kPa and 16.2±2.2 kPa for 0.5 g/mL and 0.1 g/mL, respectively). Similarly, this significant increase in YM due to concentration was also observed for aerogels (438.70±25.8 kPa and 91.36±8.2 kPa, respectively). Results (FIG. 10 A) showed a non-significant increase in the YM between the hydrogels at 0.1 g/mL medium (16.2±2.2 kPa) and the aerogels at 0.1 g/mL (91.36±8.2 kPa; p=0.45). A significant increase in the YM was between the hydrogels and aerogels at 0.5 g/mL (274.88±54.8 kPa and 438.70±25.8 kPa, respectively). The compressive strength of hydrogels was measured as maximum stress before brittle fracturing, for 0.1 g/ml and 0.5 g/mL (FIG. 10 B). Data showed a significant increase in the compressive strength of the 0.5 g/mL (74.30±29.72 kPa) hydrogels compared to the 0.1 g/mL (2.78±0.54 kPa) hydrogels. Due to their compliant nature, no compressive strength was observed in the aerogels, and deformed plastically until 100% strain.

### Cell Culture and Differentiation

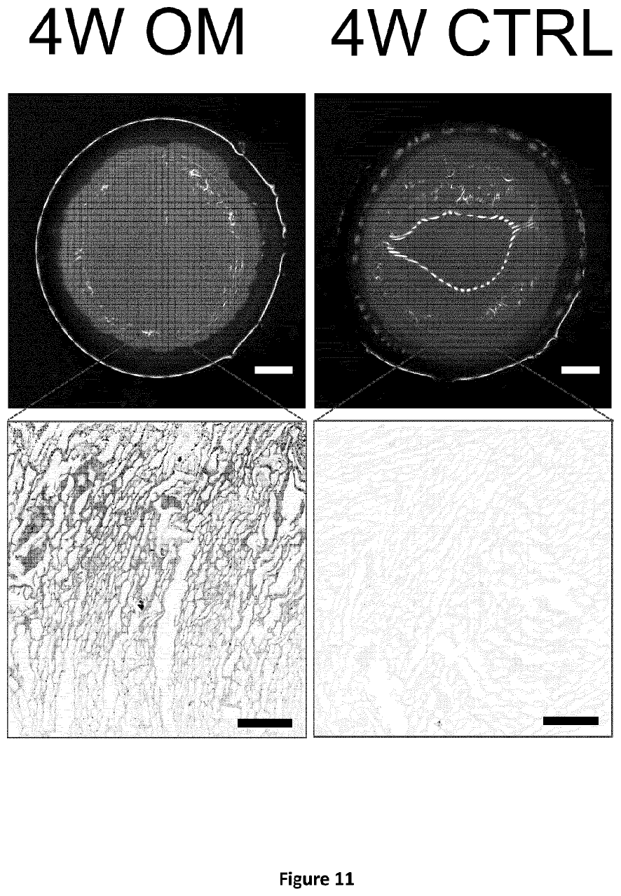

Aerogels (0.5 g/mL) were used as scaffolds for pre-osteoblast cell culture and differentiation. Prior to seeding on aerogel disks, MC3T3-E1 Subclone-4 pre-osteoblast cells (ATCC® CRL-2593™) were cultured in MEM with addition of 10% fetal bovine serum (FBS) and 1% penicillin/streptomycin (α-MEM). Cells were tryspinized and re-suspended in a 40 μL aliquot containing 106 cells. Each aliquot was pipetted on the surface of the scaffolds in a 96-well plate. Cells were left to adhere for 1 h in cell culture conditions. Then, 200 μL of α-MEM were added to each culture well. Culture medium was changed every 3 to 4 days, for 7 days. Osteoblastic differentiation of MC3T3-E1 cells was induced by adding 50 μg/mL of ascorbic acid and 10 mM β-glycerophosphate to α-MEM to obtain an osteogenic differentiation medium (OM). OM was changed every 3 to 4 days, for 4 weeks. Control group was cultured in α-MEM for the same period of time with similar medium renewal frequency. Cell-seeded aerogels cultured in osteogenic-inducing media displayed a white opaque coating, compared to the samples in regular culture media (FIG. 11). This coloring could be attributed to sign of mineralization on the constructs by the differentiation of MC3T3-E1 cells. Further confirmation of mineralization was obtained with histological sectioning of the scaffolds after 4 weeks.

Mineral deposits were assessed by histological sectioning and staining (N=3 per incubation condition). Cell-seeded aerogels were fixed after 4 weeks of incubation in either OM or α-MEM, with 10% formalin for 30 min and placed in 70% ethanol. Paraffin embedding and staining was performed as previously described. Slides were stained with Von Kossa/Van Geison (VK) for mineral deposition. Images were acquired with a Zeiss AXIOVERT 40 CFL microscope at a 40× magnification. VK staining revealed mineral deposits on pore walls of the samples incubated in OM (FIG. 11). On the contrary, no mineral deposits were visible on pores walls for samples incubated in regular culture media and were only displaying pale pink coloring (FIG. 11). Furthermore, differentiation of MC3T3-E1 cells had an impact on the YM of the aerogels.

Cube-shape printed hydrogels were constructed as previously described. Prior to seeding on hydrogels, MC3T3-E1 Subclone-4 pre-osteoblast cells (ATCC® CRL-2593™) were cultured in MEM with the addition of 10% fetal bovine serum (FBS) and 1% penicillin/streptomycin (α-MEM). Cells were tryspinized and resuspended in a 40 μL aliquot containing 106 cells. Each aliquot was pipetted on the surface of the scaffolds in a 24-well plate. Cells were left to adhere for 1 h in cell culture conditions. Then, 200 μL of α-MEM were added to each culture well. FIG. 15A shows MC3T3-E1 pre-osteoblast cells seeded on printed hydrogel cube scaffolds (Confocal imaging). FIG. 15B shows MC3T3-E1 pre-osteoblast cells seeded on printed hydrogel cube scaffolds (SEM imaging). FIG. 15C shows surface of printed hydrogel cube scaffolds (SEM imaging).

### Mechanical Analysis of Cell-Seeded Aerogels

To assess mechanical changes of the cell-seeded aerogels due to MC3T3-E1 differentiation, YM of the samples were measured after 4 weeks of incubation in either OM or α-MEM. Samples (N=3 per incubation condition) were compressed at 1%/sec rate and the resulting YM was taken by fitting the linear portion for the stress-strain curve.

Results depicted in FIG. 12 showed significant increase of the YM between the samples cultured in OM (78.29±19.46 kPa) and the samples cultured in α-MEM (CTRL) (17.66±3.55 kPa). Mineralisation of the scaffolds did have a significant influence on the YM of the hydrogels. Further investigation of the MC3T3-E1 differentiation was performed with Alizarin Red S (ARS) staining and Energy-dispersive spectroscopy (EDS) analysis

### Mineralization Analysis of Cell-Seeded Aerogels

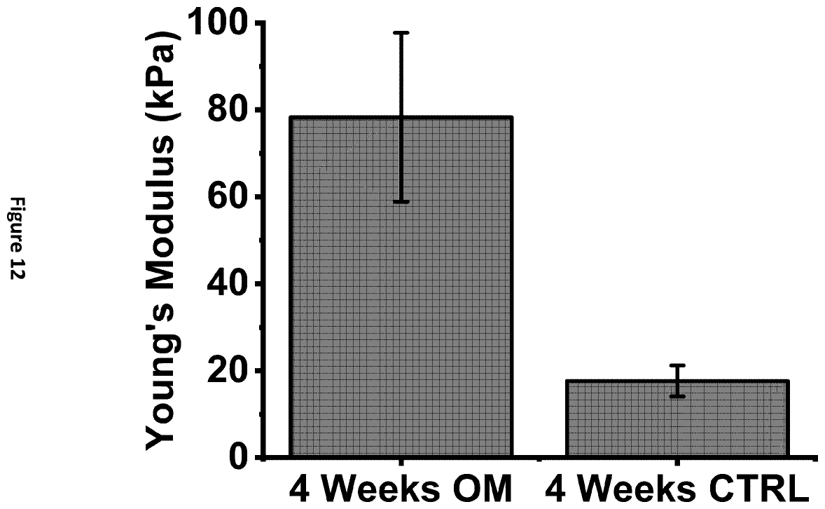

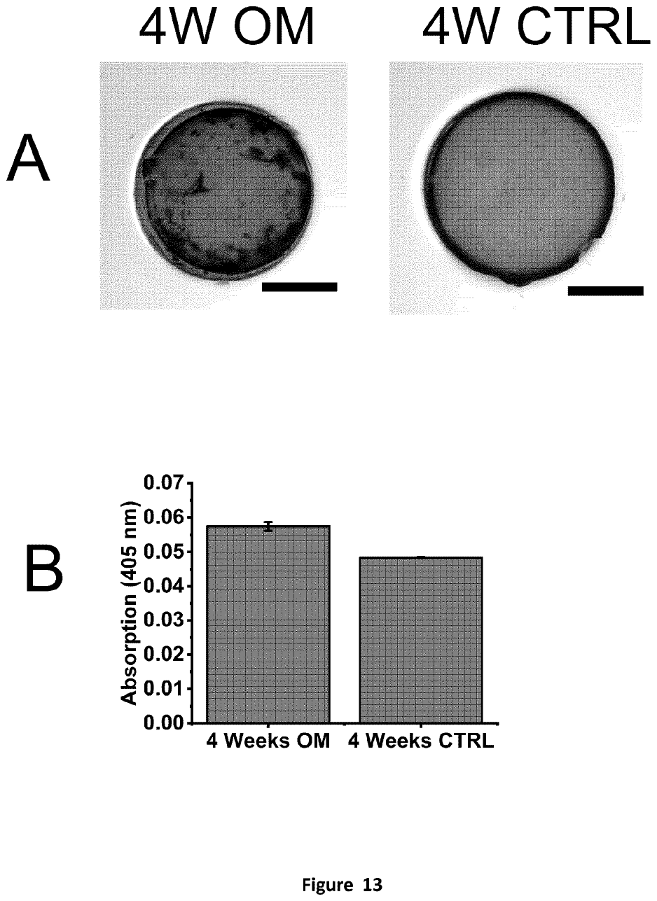

Mineralization of the cell-seeded aerogels were assessed by ARS and EDS after 4 weeks of incubation in either OM or α-MEM. Cell-seeded aerogels, after 4 weeks of incubation in either OM or α-MEM (N=3 per incubation condition) were fixed with 10% formalin for 30 min and washed with deionized water. Thereafter, samples were stained with a 2% ARS (pH=4.1) solution for 45 min at room temperature. Samples were then thoroughly washed with deionized water to remove excess staining and placed in 15 mL conical tubes with 10 mL of deionized water. Tubes were placed on an orbital shaker at 120 rpm for 1 h, periodically renewing dH₂O every 15 min. Samples were imaged with a Nikon SMZ1270 stereomicroscope with dark-red staining indicating calcium deposits. Thereafter, ARS stained samples were processed for optical calcium quantification, following established protocol (15). Briefly, stained samples were incubated in 800 μL of 10% acetic acid solution for 30 min with light agitation. The solution was transferred to 1.5 mL tubes and centrifuged at 17×104 g for 15 minutes and 500 μL of the supernatant was collected and transferred to a new tube with 200 μL of 10% ammonium hydroxide. From this solution, 150 μL was collected and transfer to a 96-well plate. Absorption at 405 nm was performed with an automated plate reader, with each sample analysed in triplicate. Aerogels cultured in OM displayed strong, dark red coloration with ARS staining on localised areas of the scaffolds (FIG. 13 A). The other areas of the scaffolds stained also in red, but with less opacity. On the contrary, aerogels cultured in regular culture media did not displayed dark-red coloration after ARS staining (FIG. 13 A). Mineral deposition was further evaluated by eluting the ARS-stained aerogels in an acetic acid solution. Results showed that samples incubated in OM had a significantly higher mineral content than samples incubated in α-MEM after 4-weeks (FIG. 13B, p=2×10⁻⁶).

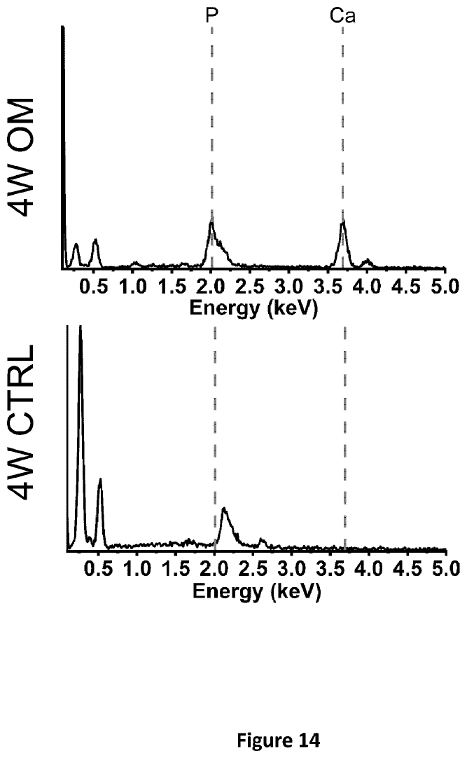

Moreover, cell-seeded aerogels (N=3 per incubation condition) were fixed with 10% formalin for 30 min and dehydrated in increasing concentration of ethanol (from 70% to 99%). Samples were processed in a critical-point dryer and were coated with a 9 mm layer of gold. Surface analysis of the samples was obtained with Energy-dispersive spectroscopy (EDS) to observe the presence of Phosphorus (P) and Calcium (Ca). Samples cultured in OM displayed characteristic emission signals for phosphorus (2.0134 keV, P) and calcium (3.6905 keV, Ca) (FIG. 14). P and Ca signals were not observed for samples cultured in α-MEM, indicating that MC3T3-E1 were mineralizing the aerogels only when incubated in OM. These results on mineralisation revealed that mCNF aerogels can support osteoblastic differentiation.

### Statistical Analysis

Data are reported as mean±standard error of the mean. One-way ANOVA was performed as statistical followed by Tukey post-hoc tests for YM mean comparison at different concentration. Two-sample T-test was performed for Compressive Strength and mineralisation mean comparison. A value of p<0.05 was considered to be statistically significant.

### Bone Filler

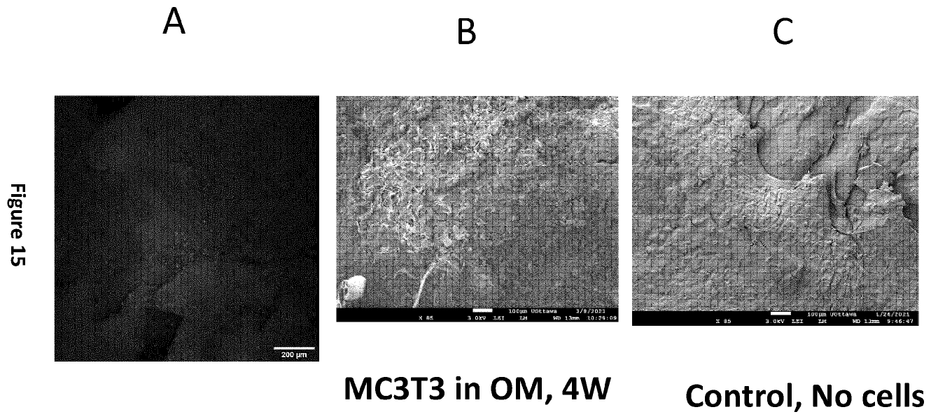

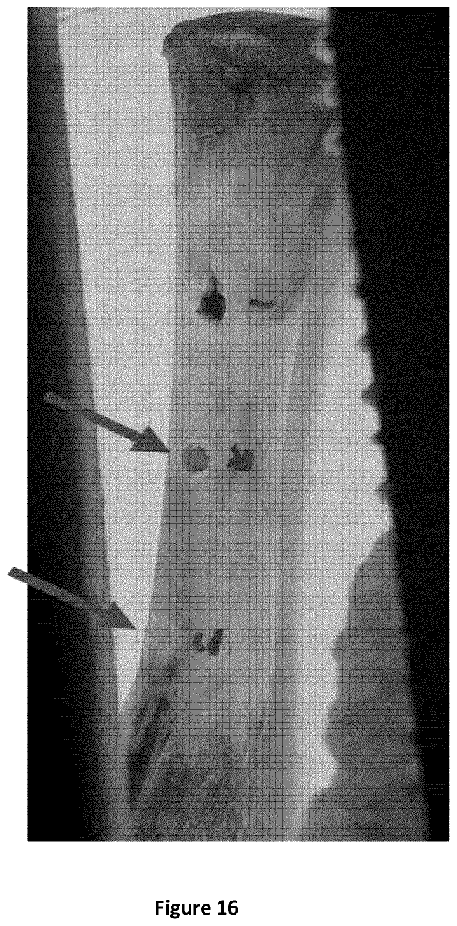

Methacrylated cellulose nano fibrils resin was pipetted in perforated holes in a chicken femur bone. The surface of the bone was then illuminated with 405 nm UV light. FIG. 16 shows bone glue (red arrows) produced with methacrylated cellulose nano fibrils resin under UV light for curing.

### Cell Culture Dish Coating

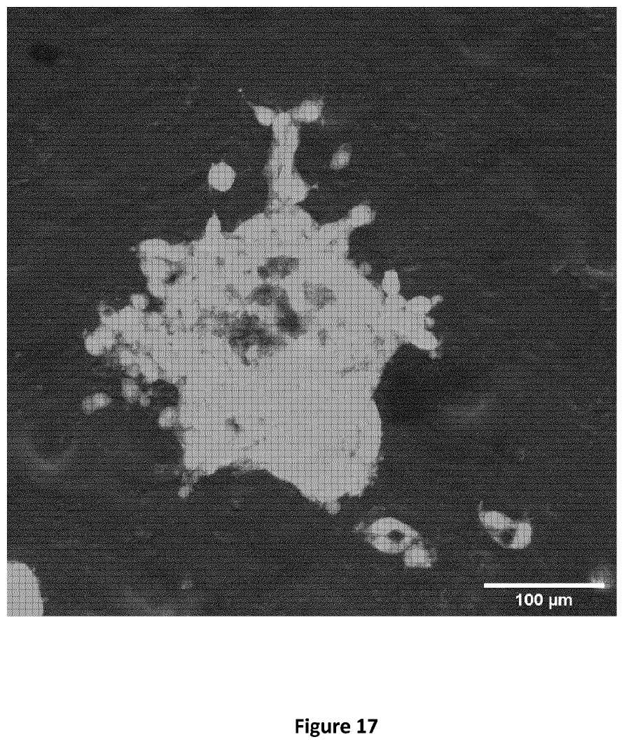

Methacrylated cellulose nano fibrils resin was pipetted in wells of 24-well plate culture dish and the surface was illuminated with 405 nm UV light. GFP cell suspension was pipetted on the coated surface. FIG. 17 shows GFP-3T3 cells on a resin-coated culture dish.

### Material Surface Coating

Methacrylated cellulose nano fibrils resin was pipetted on surface of different materials: Aluminum, wood and stainless steel. FIG. 18 shows different materials coated with mCNF resin (SEM imaging).

Several Applications of Cellulose have been Explored which Include:
“Bio-Ink”: for Tissue engineering;
Scaffolds of different pore size/geometry for in vitro cell culture: Scaffolds are printed with desired geometry (e.g. cubes), sterilized and seeded with different cell types (e.g. MC3T3; GFP-3T3). Cells proliferate on/in the scaffolds. Differentiation can be induced;
Scaffolds for in vivo implantation: Scaffolds are printed with desired geometry (e.g. cubes), sterilized. Scaffolds are implanted;
Bone “glue”/filler for implant-bone or bone-bone adhesion: The uncured “ink” is injected into the defect then exposed to UV light until cross-linking;
Dental cavity repair (In situ cross-linking): The uncured “ink” is injected into the dental cavity then exposed to UV light until cross-linking;
Bone/Dental implant: The implant is printed with desired geometry and shape before being sterilized and implanted;
Implant for tissue repair (Skin, cartilage, muscle, etc.): The implant is printed with desired geometry and shape before being sterilized and implanted;
Implant for esthetic purposes (e.g. breast, nose, ear): The implant is printed with desired geometry and shape before being sterilized and implanted;
Microfluidic devices (micro bioreactors, solvent mixing, cell separation, cell encapsulation): The device is printed with desire geometry/shape. Cells are seeded in the device;
Microcarrier for suspension cell culture;
“Ink” for UV curing resin-based 3D printers and for extrusion 3D printers with UV cross-linking post-print;
Additive for UV curable paint and clear finish;
Additive for resins and glues (e.g. Epoxy);
UV curable hardener for resins (e.g. Epoxy floor covering);
UV curable Nail polish;
Absorption substrate;
Automotive coatings;
Plastics coating;
Wood coating;
Circuit board coating;
Conformal coating;
Optical coating;
Furniture and wood coatings;
Industrial coatings;
Glue/filling for bone and dental repair; and
Wound dressing/closure.
Cardiac stent coating;
Glass, screen lamination;
Protective layer for wood; and
Coating for modulating hydrophobicity.

### Specific Applications

### Wood Coating

In the wood coating experiments, the potential of incorporating mCNF (made from reacting aminoCell with different chemical modifiers: methacrylic anhydride or glycidyl methacrylate to forming a functional coating formulation was evaluated. These modifiers were dispersed in a base formulation, which served as a control. The exemplary base formulation consisted of:
30% v/v: 1,6-hexanediol diacrylate
65% v/v: Aliphatic urethane acrylate (EBECRYL 4680 from Allnex Inc).
5% v/v: Darocur 1173, as photoinitiator, was included to facilitate the curing process through exposure to appropriate light sources.

To the 1 g of base formulation, approximately 0.01 g of mCNF (via GMA) or mCNF (via MA) was dispersed at high RPM with a stir bar (>1200 RPM). Then, the solution was placed in a vacuum chamber for at least 1 h to remove bubbles.

One drop of the solution was placed on a piece of medium density fiberwood and carefully smeared with a glass slide. The coated surface was exposed with 405 nm UV light for 30 sec. This procedure was repeated 5 times, until fully cured.

“Scrub test”: Using an ethanol-soluble blue marker, the letters “SCT” were written on coated uncoated parts of the board (FIG. 39). Using a 70% ethanol-soaked wipe, both sections were vigorously scrubbed for 10 seconds. This resulted in the ink (on the uncoated part) to diffuse through the board and wasn't absorbed by the scrubbing wipe. On the contrary, on the coated section, the ink did not diffuse into the underlying wood board and was removed by the scrubbing wipe. The ethanol was let to evaporate for 5 min, after the scrubbing experiment. As a result, a deformation (bulging) of the uncoated part was observed, whereas the coated part remained intact.

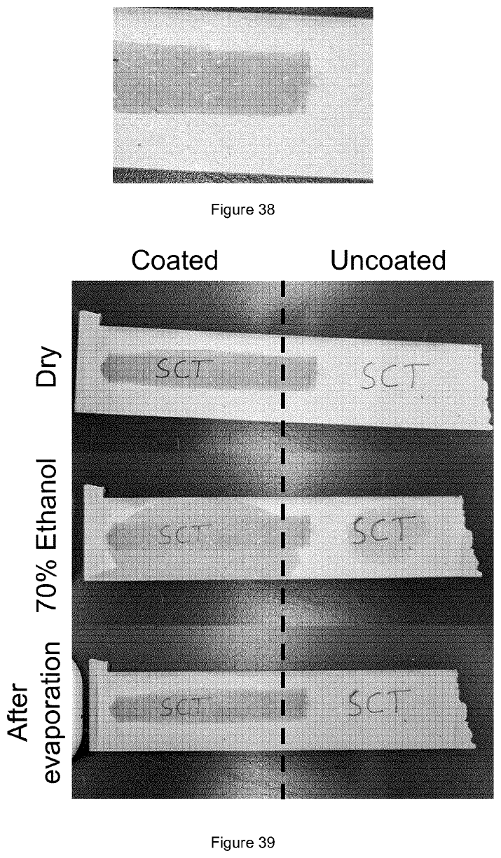

The mCNF could be lyophilized prior to dispersion in the “base formulation”. It was observed that lyophilized mCNF rapidly dissolves under high shear, whereas non-lyophilized mCNF does not. Therefore, a different dispersing method (i.e., homogenizer) may be required to disperse non-lyophilized mCNF into base formulation. The coated Medium Density fiber wood with the mCNF (via MA)-containing formulation—5 coats with 30 sec UV exposure is shown in FIG. 38. The coated Medium Density fiber wood with the mCNF (via MA)-containing formulation “scrub test” is shown in FIG. 39. The coated Medium Density fiber wood with the mCNF (via GMA)-containing formulation—5 coats with 30 sec UV exposure is shown in FIG. 40.

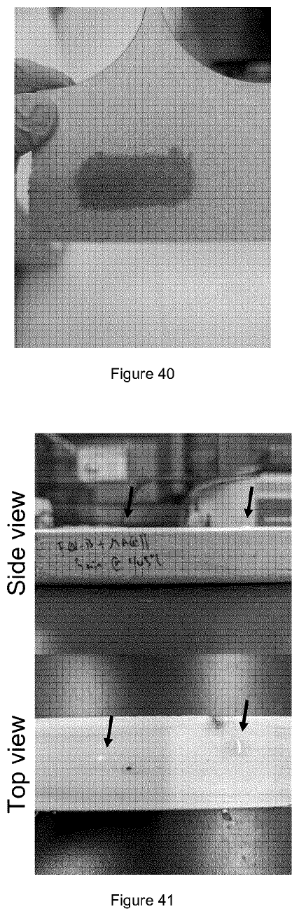

Separately, a few drops of the mCNF-containing solution was placed on a piece of sanded wood and carefully smeared with a glass slide. The coated surface was exposed with 405 nm UV light for 5 min. Droplets of water were pipetted on the coated surface (FIG. 41) and it was observed that the surface appeared hydrophobic. The coated sanded wood with the mCNF-containing formulation—1 coat with 5 min UV exposure is shown in FIG. 41. Note arrows are pointing to water droplets.

### PCB Conformal Coating

In the wood coating experiments, the potential of incorporating mCNF (obtained using MA) to a functional coating formulation was investigated. The modifier was dispersed in a base formulation, which served as a control. The base formulation consisted of:
87.2% v/v: Isononyl Acrylate
8.2% v/v: Aliphatic urethane acrylate (EBECRYL 4680 from Allnex Inc).
4.6% v/v: Darocur 1173, as photoinitiator, was included to facilitate the curing process through exposure to appropriate light sources.

To the 4.739 g of base formulation, approximately 0.05 g of mCNF (via MA) was dispersed at high RPM with a stir bar (>1200 RPM). Then, the solution was placed in a vacuum chamber for at least 1 h to remove bubbles.

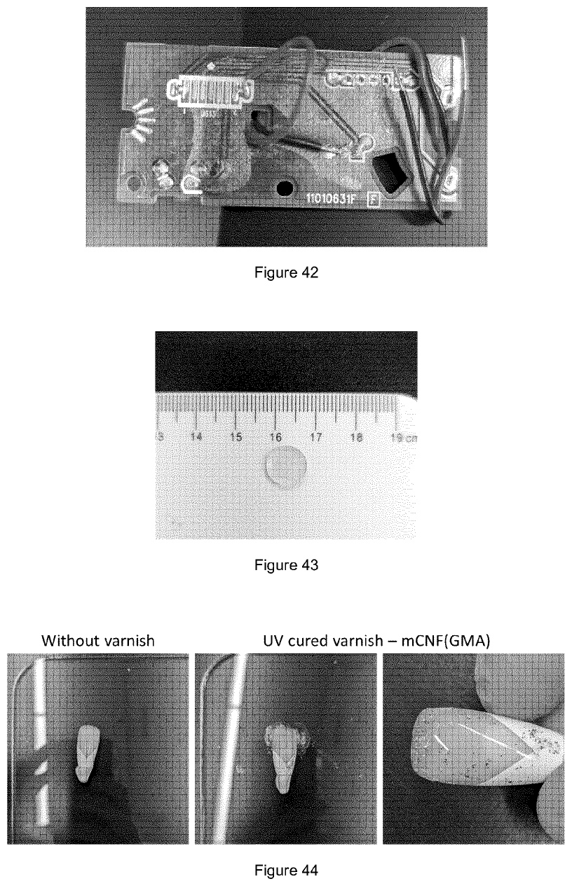

The solution was poured over an unprotected printed circuit. The coated surface was exposed with 405 nm UV light for 3 min. This resulted in a hard layer over the circuit board. The circuit board was washed with excess was and tested for electrical conductivity. With a multimeter, voltage testing indicated insulation of the circuit as no voltage was detected when probing over the coated surface. The coated printed circuit board with the mCNF (via MA)-containing formulation—1 coat with 3 min UV exposure is shown in FIG. 42.

3D Shaping of the mCNF Resin

In the 3D shaping experiments, mCNF (via GMA) were incorporated to a functional resin formulation. These modifiers were dispersed in a base formulation, which served as a control. The base formulation consisted of:
30% v/v: 1,6-hexanediol diacrylate
65% v/v: Aliphatic urethane acrylate (EBECRYL 4680 from Allnex Inc).
5% v/v: Darocur 1173, as photoinitiator, was included to facilitate the curing process through exposure to appropriate light sources.

To the 5.03 g of base formulation, approximately 0.1052 g of mCNF (via GMA) was dispersed at high RPM with a stir bar (>750 RPM). Then, the solution was placed in a vacuum chamber for at least 1 h to remove bubbles. The solution was pipetted into a 6 mm (diam.) by 2 mm (thickness) cylindrical Teflon mold and exposed to 405 nm UV light for 5 min. It is noted that the resin could also be used for DLP (Digital Light Processing) 3D printing of constructs. The casted and cured disk made with the mCNF (via GMA)-containing formulation and 5 min UV exposure is shown in FIG. 43.

### UV Cured Nail Polish Varnish

In this experiment with UV curable nail polish formulation, the potential of incorporating mCNF synthesized via glycidyl methacrylate (mCNF (via GMA)) was investigated. The formulation consisted of the following ingredients:
Isobornyl acrylate: 90.09% w/w
mCNF (via GMA): 0.90% w/w
Acrylated urethane (EBECRYL): 8.79% w/w
Glitter: 0.25%

A small coat of the formulated mixture was applied to an artificial nail with a paint brush. Subsequently, the coated surface was exposed to 405 nm UV light for a duration of 5 minutes. As a result of this UV exposure, a cured layer formed over the nail, indicating the successful polymerization and hardening of the formulation. UV curable nail polish formulation including mCNF (via GMA) is shown in FIG. 44 wherein a small coat of the formulated mixture was applied to an artificial nail with a paint brush and exposed to 405 nm UV light for 5 minutes.

### Example 2—BY2 Cells

In this example, an alternative cellulose source was explored by utilizing decellularized BY-2 Tobacco cells, rather than CNF (microfibrillated cellulose). To obtain the desired modified cellulose, a reaction route that involved: cellulose to tosylCell; tosylCell to aminoCell; aminoCell to mCNF (via glycidyl methacrylte (as previously described)) was used.

This experiment confirmed the feasibility of utilizing different sources of cellulose, such as sawdust or cellulosic waste, for the synthesis of methacrylated cellulose derivatives.

UV curing of methacrylated BY-2 tobacco cell.
1. The resulting solid (following methacrylation protocol) was dissolved in water at a 1:20 mass ration.
2. Lithium phenyl-2,4,6-trimethylbenzoylphosphinate (LAP) was added at 0.2% w/v.
3. 50 μL of the solution was pipetted in cylindrical Teflon mold (6 mm diam.×2 mm thickness).
4. Optionally, unmodified decellularized BY-2 tobacco cells can be added to the solution (as a dispersion)
5. A step of photo-crosslinking is carried out under 405 nm light for 10 min.
6. Disks are frozen overnight at −80 C and lyophilized for 24 h.
7. GFP-3T3 are cultured onto the disks over an 11-day period.

The confocal images of culture of GFP-3T3 over an 11-day period of (A) methacrylated BY-2 tobacco cell and (B) methacrylated BY-2 tobacco cell with the addition of unmodified decellularized BY-2 tobacco cells is shown in Figure XX A and Figure XX B.

### Example 3—Pectin

The global citrus pectin market was valued at US$547.2 Mn in 2018, and is being used by industries such as pharmaceuticals, and cosmetics & personal care. Pectin is a plant cell wall polysaccharide that constitutes soft or growing tissues of dividing cells and is the matter of ~35% of the dicocotyledon cell walls. Pectin is one of the most complex polysaccharides, the structure of which combines a linear homopolymeric region of a-(14)-Galacturonic acid that corresponds to 65% of the overall polysaccharide, with mainly two segments of heteropolysaccharides highly substituted, constituted by Arabinose, Galactose, and Rhamnose (16). Referred to as RG-I and RG-II, these Rhamnogalacturans regions vary in the content of fucose, galactose (RG-I), L-aceric acid and D-apiose (RG-II) (17). The structure of pectin polysaccharides are depicted, showing the main substructures: homogalacturonan (HG), rhamnogalacturonan I (RGI), rhamnogalacturonan II (RGII), xylogalacturonan (XGA) and apiogalacturonan (18).

The carboxylic acid of the galacturonan residues can be 6-O-methylated or 2- and/or 3-O-Acetylated. The degree of esterification (DE) is related to the methylated groups only and is used to classify pectins for commercial uses. The DE depends on the source (16) and can be affected by the right control of pH, temperature and time of the extraction process.

Similar to other acidic polysaccharides, pectin produces stable gels with divalent cations, e.g. Ca²⁺, but near to 20 consecutive blocks of unmethylated GalA residues are necessary to obtain the cross-linking (20).

Pectin is found virtually in all plants, but commercially are obtained from citrus fruits: orange, lemons, grapefruit, and apples, generally derived from juice production.

The citrus industry mainly produces Juice/pulp 45-55%, Peel 45-55% and essential oil 0.2-0.5%. Generally, 10-20% of pectin can be extracted from dry Apple pomace, Sugar beet (10-20%), Sunflower (15-25%), but citrus peel is the largest available source and present a yield of 25-35% from the dry fruit (19,21).

Pectin with low DE such as the pectin found in limes and lemons are typically preferred food industry due to better gel behavior and higher specific viscosity compared to the pectin found in oranges and grapefruits which has higher rates of methyl and acetyl esters and lower viscosity. Indeed, pectin found in limes and lemons present higher specific viscosity and may form gels in a wider pH range compared to the pectin found in oranges and grapefruits (22-24). In other words low DE pectins are considered as high quality grade suitable as additives.

Pectins will aggregate and produce gels in mixtures with proteins, or with pure pectin solutions with Ca²⁺ or not, depending on DE, temperature and pH (25).

Pectin is used in various products as emulsifiers and gelling agents for jams, low fat creams (fat free mayonnaise, cheese, desserts, yogurts and sausage stuffing), stabilizers for pharmaceutical material and personal care products (26, 27).

A summary of the applications of pectin are summarized in Table 1 (28):

| Food and nutrition industry | Therapeutic purposes | Tissue engineering |
|---|---|---|
| Prebiotic potential and | Constipation | Tissue scaffold |
| probiotic carrier | Immune response | |
| Texturizer and organoleptic | modulator | |
| sense improver in dairy | Drug delivery | |
| products | Cancer therapy | |
| Encapsulation | Wound healing | |
| Lower fat absorption in | | |
| potato chips | | |
| Food packaging | | |
| 3D printed food | | |
| Control of dietary uptake | | |

Most of the chemical modifications focus on reducing the degree of esterification, during the extraction process or after, to obtain low methoxyl (LM) pectin with DE<50% from high methoxyl pectins with (HM, DE>50%).

An example of a method of transforming pectin into cross-linkable pectin will now be described. Pectin was dissolved in dH₂O at 4% w/v. Once the polysaccharide is fully dissolved, 10% v/v of methacrylic anhydride is added dropwise with vigorous stirring, while maintaining the pH between 8 and 8.5 with 5N sodium hydroxide. The solution is stirred at room temperature for 10 min. The solution was kept for 24 h at 5° C. under constant stirring. Afterwards, the solution is precipitated in −15° C., 99% ethanol and centrifuged at 4000 rpm for 10 minutes. The precipitate was rinsed 3 times with 99% ethanol and dialysed against dH₂O for 48 h. The precipitate was rinsed with deionized water and lyophilised overnight. FIG. 19 is a FTIR spectra of methacrylated pectin compared to pristine pectin.

### Nuclear Magnetic Resonance Spectroscopy

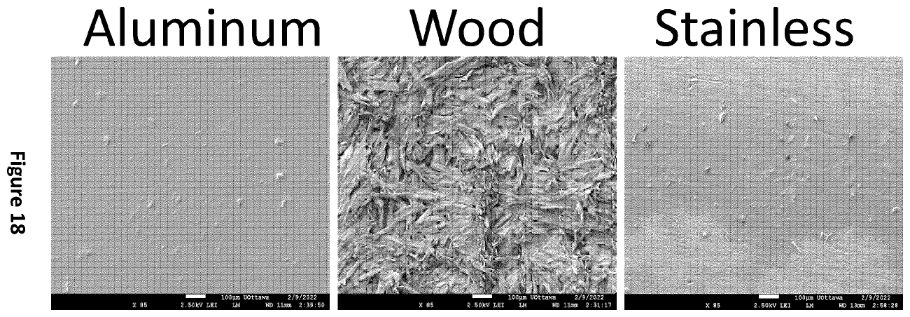

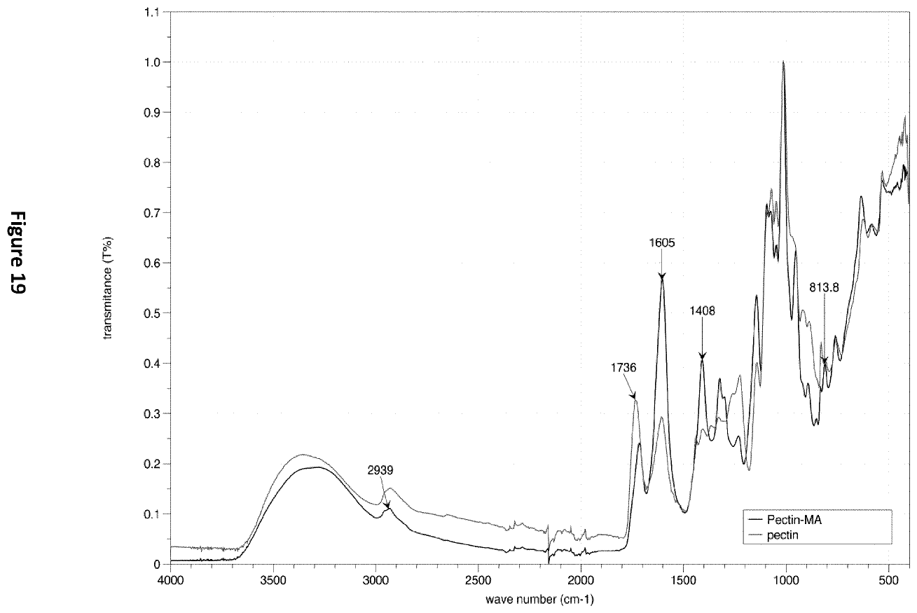

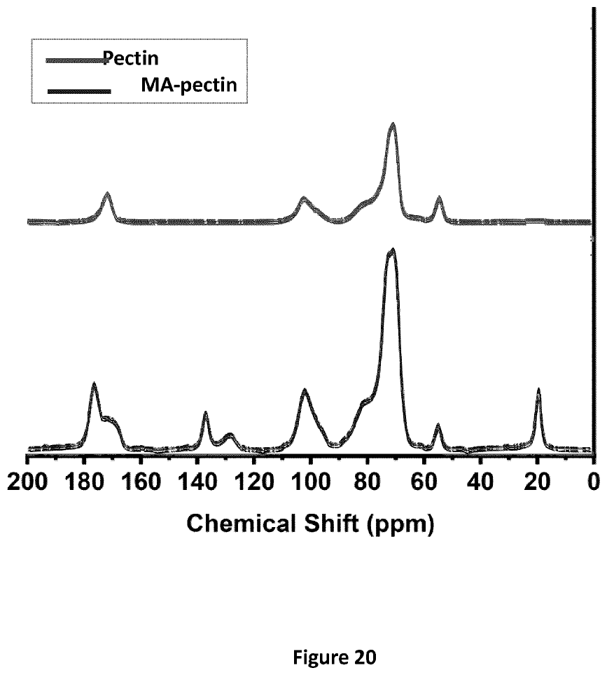

Solid state Carbon-13 Magic Angle Spinning (MAS) NMR spectra were recorded with a Bruker Avance III 200 (Bruker, Billerica, Massachusetts/USA). Spectra were collected at a 200 MHz frequency. Samples were grinded to a fine powder using a mortar and pestle. FIG. 20 shows a ¹³C solid state NMR spectra of methacrylated pectin compared to pristine pectin.

Pectin modification with methacrylate anhydride in semi-heterogeneous condition is seen in FIG. 20 with ¹³C solid state NMR. Spectra of pristine pectin is showing characteristic carbon chemical shifts at 101 ppm for the anomeric signal C-1, 68 ppm for C-2, C-3, C-5, 80 ppm to C-4, the carbonil C-6 at 171 ppm. The methoxy group (O-Methyl) and CH3 appear respectively at 53 ppm and 20 ppm. After chemical modification, new peaks appeared corresponding to the vinyl carbons (=CH2) at ~135 ppm, as well the quaternary carbon at ~145 ppm, indicating substitution of the acidic group in the pectin by methacrylate group.

Transforming Cross-Linkable Pectin into Light Cross-Linkable Pectin

In order to transform the cross-linkable pectin into light cross-linkable pectin, methacrylated pectin was dissolved in 5 mL of dH2O to 3% and vehicle q.s. 0.1% of LAP (bis(acyl)phosphane oxi lithium phenyl-2,4,6-trimethylbenzoyl phosphinate) was added to generate a light cross-linkable pectin solution.

UV Curing: Transforming Light Cross-Linkable Pectin into a Resin

In order to transform the light cross-linkable cellulose into a resin, the light cross-linkable pectin solution was exposed under 405 nm light, 6 to 10 W, ~30 mW/cm2 for intervals of 5 minutes.

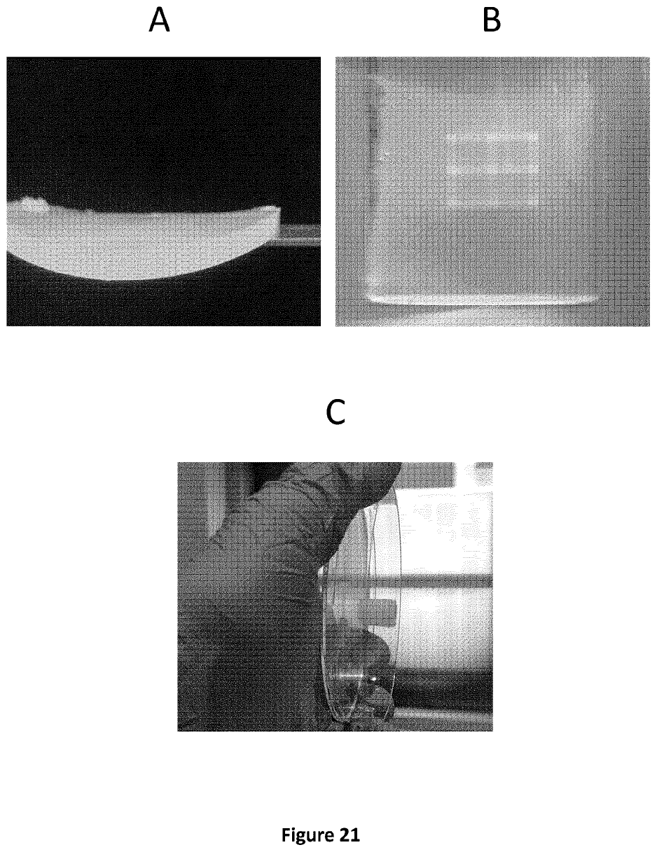

The methacrylated pectin, when submitted to UV light at 405 nm in presence of LAP, changes to a rigid but breakable solid that can be seen on FIG. 21A. Constructs can be produced by stereolithography (FIG. 21B): a grid with 10 mm×10 mm with inner lines having a thickness of 1 mm. The time of exposure was adjusted in order to not crosslink the resin that surround the irradiated region.

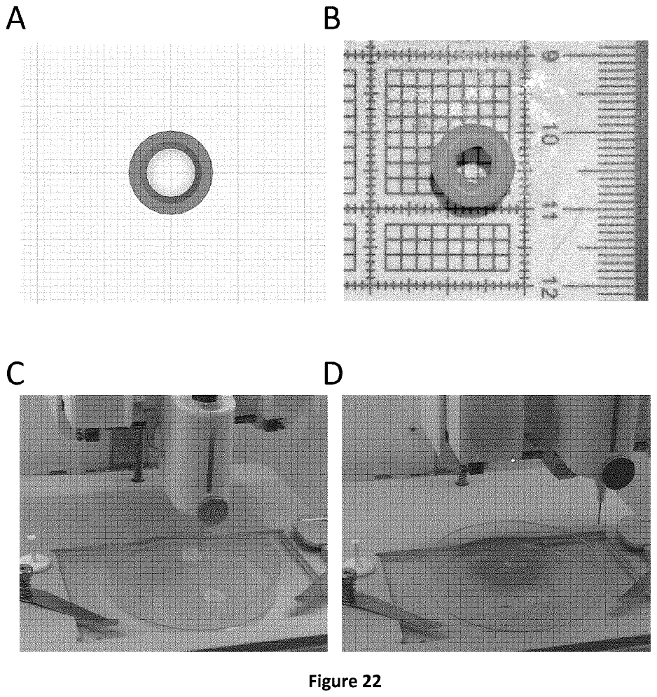

The additive printed construct, a cylinder with 10 mm wide, and 20 mm high shown in FIG. 21C, and with a thickness of 2.5 mm that can be seen on FIG. 22B, after sequential deposition of layers of the mixture of the commercial resin composed by alginate, cellulose and mannitol with the methacrylated pectin in the presence of the photoinitiator, LAP.

### 3D Printing

Constructs such as the cylinder with dimensions of 10 mm wide, 20 mm high, and 2.5 mm for the wall thickness (FIG. 22A) were generated and printed (FIGS. 22B, C and D) using the BIO X Bioprinter (Cellink Inc., Boston, Massachusetts/USA). The resin was prepared with 0.1 g of methacrylated pectin and 0.01 g of LAP was mixed with 3 mL of commercial resin composed by alginate, cellulose and mannitol. During the printing each layer was exposed for 10 sec of UV light at 405 nm. After printing, the construct where washed with water many times than reserved on 70% ethanol. FIG. 16 shows an example of a cylinder printed with methacrylated pectin resin

### UV Stereolithography 3D Printing

Constructs (FIG. 21B) were printed using the LumenX DLP Bioprinter (Cellink Inc., Boston, Massachusetts/USA). The resin solution in dH₂O was prepared with 1 to 3% of methacrylated pectin and 0.5% of LAP. 1 mL of the final printing solution with the photo initiator was pipetted in the bioprinter and each layer was exposed for 3 sec at 60% of UV power. FIG. 21B shows an example of printing by stereolithography using methacrylated pectin.

### Cell Seeding

After washed the constructs were places in a 6-well plates with DMEM high glucose (Dulbecco's Modified Eagle's medium high glucose) containing 10% BSA and 1% penicillin/streptomycin and was than seeded to with mouse myoblast cells C2C12 culture. The cells were incubated in 5% CO2 at 37° C., changing the medium every 2 days. The myogenic differentiation was induced with 2% horse serum DMEM/1% penicillin/streptomycin, after a period of two weeks to allow cells grow to confluence. The C2C12 cells were treated with 3.5% paraformaldehyde and permeabilized with Triton X-100, and stained with TRITC-phalloidin (Sigma-Aldrich, USA) and DAPI (Thermo Fisher Scientific, USA).

### Staining and Confocal Laser Scanning Microscopy

Cells were fixed with 3.5% paraformaldehyde and permeabilized with Triton X-100. Thereafter, cells were with Alexa Fluor 594 phalloidin (PH-594, Sigma-Aldrich, USA) and DAPI (Thermo Fisher Scientific, USA) to highlight actin fibers and DNA, respectively. Samples were imaged with confocal laser scanning microscope (Nikon Ti-E A1-R) equipped with a 10× objective. Confocal images were created by maximum projection in the Z axis using ImageJ software.

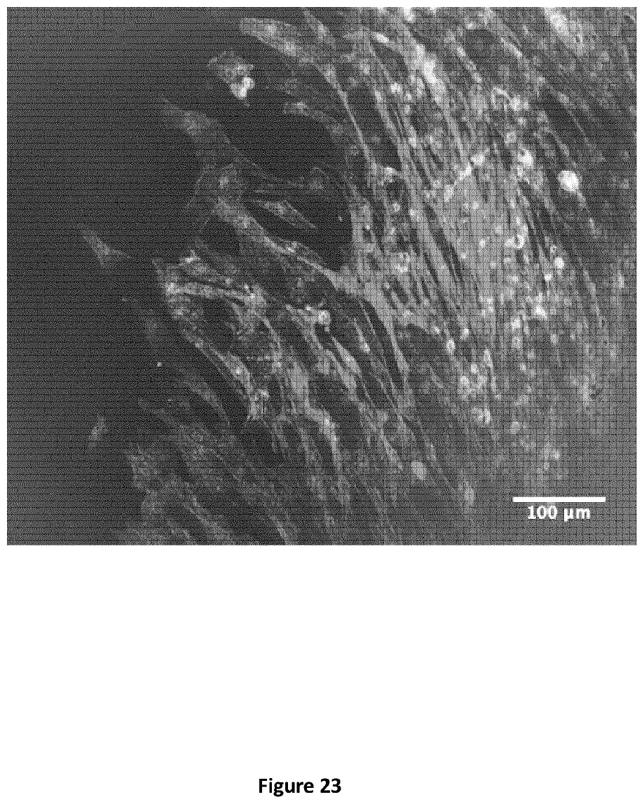

A cylinder printed with additive technique was seeded with C2C12 mouse myoblast cells and allow to differentiate for 45 days. Cells stained with PH-594 can be seen surrounding the construct after (FIG. 23, red staining). The material was compatible with development of mammalian cells.

Taken together, these analyses show that pectin can be used in photo-crosslinkable inks in solution for stereolithography as well in additive printing mixed with regular thickener polysaccharides, generating 3D constructs that are biocompatible.

While illustrative and presently preferred embodiments of the invention have been described in detail hereinabove, it is to be understood that the inventive concepts may be otherwise variously embodied and employed and that the appended claims are intended to be construed to include such variations except insofar as limited by the prior art.

### REFERENCES

1. T. Li et al., “Developing fibrillated cellulose as a sustainable technological material,” Nature, vol. 590, no. 7844, pp. 47-56, February 2021, doi: 10.1038/s41586-020-03167-7.
2. N. S. Sharip and H. Ariffin, “Cellulose nanofibrils for biomaterial applications,” Materials Today: Proceedings, vol. 16, pp. 1959-1968, January 2019, doi: 10.1016/J.MATPR.2019.06.074.
3. J. Lee, H. Jung, N. Park, S. H. Park, and J. H. Ju, “Induced Osteogenesis in Plants Decellularized Scaffolds,” Sci. Rep., vol. 9, no. 1, pp. 1-10, December 2019.
4. US20200262802, “Photo-crosslinked hydrogel material and preparation, composition, and application thereof photo-crosslinked hydrogel.” [Online]. Available: https://patentscope.wipo.int/search/en/detail.jsf?docId=US302853008&docAn=16848372
5. WO2009155583A1, “Biomaterials for tissue replacement—Google Patents.” [Online]. Available: https://patents.google.com/patent/WO2009155583A1/en?oq=biomaterials+for+tissue+replacement+trustees+pensilvania.
6. WO2019173637A1, “Nanocellulose-containing bioinks for 3d bioprinting, methods of making and using the same, and 3d biostructures obtained therefrom—Google Patents.” [Online]. Available: https://patents.google.com/patent/WO2019173637A1/en.
7. S. Schmidt, T. Liebert, and T. Heinze, “Synthesis of soluble cellulose tosylates in an eco-friendly medium,” Green Chem., vol. 16, no. 4, pp. 1941-1946, March 2014.
8. P. B. Groszewicz et al., “N-Hydroxysuccinimide-activated esters as a functionalization agent for amino cellulose: synthesis and solid-state NMR characterization,” Cellulose, vol. 27, no. 3, pp. 1239-1254, February 2020.
9. D. J. Modulevsky, C. M. Cuerrier, and A. E. Pelling, “Biocompatibility of Subcutaneously Implanted Plant-Derived Cellulose Biomaterials.,” PLoS One, vol. 11, no. 6, p. e0157894, 2016.
10. D. J. Modulevsky, C. Lefebvre, K. Haase, Z. Al-Rekabi, and A. E. Pelling, “Apple derived cellulose scaffolds for 3D mammalian cell culture.,” PLoS One, vol. 9, no. 5, p. e97835, January 2014.
11. R. J. Hickey, D. J. Modulevsky, C. M. Cuerrier, and A. E. Pelling, “Customizing the Shape and Microenvironment Biochemistry of Biocompatible Macroscopic Plant-Derived Cellulose Scaffolds,” ACS Biomater. Sci. Eng., vol. 4, no. 11, pp. 3726-3736, March 2018.
12. G. Fontana et al., “Biofunctionalized Plants as Diverse Biomaterials for Human Cell Culture,” Adv. Healthc. Mater., vol. 6, no. 8, p. 1601225, April 2017.
13. J. R. Gershlak et al., “Crossing kingdoms: Using decellularized plants as perfusable tissue engineering scaffolds,” Biomaterials, vol. 125, pp. 13-22, May 2017.
14. D. J. Modulevsky et al., “Plant Scaffolds Support Motor Recovery and Regeneration in Rats after Traumatic Spinal Cord Injury,” bioRxiv, p. 2020.10.21.347807, October 2020.
15. C. A. Gregory, W. G. Gunn, A. Peister, and D. J. Prockop, “An Alizarin red-based assay of mineralization by adherent cells in culture: Comparison with cetylpyridinium chloride extraction,” Anal. Biochem., vol. 329, no. 1, pp. 77-84, June 2004.
16. Mohnen D. Pectin structure and biosynthesis [Internet]. Vol. 11, Current Opinion in Plant Biology. Elsevier Current Trends; 2008 [cited 2021 Apr. 6]. p. 266-77. Available from: https://www.sciencedirect.com/science/article/pii/S1369526608000630
17. Lara-Espinoza C, Carvajal-Millán E, Balandrán-Quintana R, López-Franco Y, Rascón-Chu A. Pectin and pectin-based composite materials: Beyond food texture [Internet]. Vol. 23, Molecules. Multidisciplinary Digital Publishing Institute (MDPI); 2018 [cited 2021 Apr. 6]. Available from: http://www.ncbi.nlm.nih.gov/pubmed/29670040
18. Harholt J, Suttangkakul A, Scheller H V. Biosynthesis of pectin. Plant Physiol [Internet]. 2010 Jun. 3 [cited 2021 Apr. 7]; 153(2):384-95. Available from: https://academic.oup.com/plphys/article/153/2/384-395/6109523
19. Ciriminna R, Fidalgo A, Delisi R, Tamburino A, Carnaroglio D, Cravotto G, et al. Controlling the Degree of Esterification of Citrus Pectin for Demanding Applications by Selection of the Source. ACS Omega [Internet]. 2017 Nov. 30 [cited 2021 Apr. 6]; 2(11):7991-5. Available from: https://pubs.acs.org/doi/10.1021/acsomega.7b01109
20. Braccini I, Perez S. Molecular basis of Ca2+-induced gelation in alginates and pectins: The egg-box model revisited. Biomacromolecules [Internet]. 2001 [cited 2021 Apr. 6]; 2(4):1089-96. Available from: https://pubs.acs.org/doi/10.1021/bm010008 g
21. Tanhatan A, Thibault N J. Citrus pectin: structure and application in acid dairy drinks Citrus Pectin: Structure and Application in Acid Dairy Drinks [Internet]. Global Science Books; 2015 [cited 2021 Apr. 6]. p. 60-70. Available from: http://www.globalsciencebooks.info/Online/GSBOnline/images/0812/TFSB_2(SI1)/TFSB_2(SI1)60-70o.pdf
22. Morris E R, Gidley M J, Murray E J, Powell D A, Rees D A. Characterization of pectin gelation under conditions of low water activity, by circular dichroism, competitive inhibition and mechanical properties. Int J Biol Macromol [Internet]. 1980 [cited 2021 Apr. 6]; 2(5):327-30. Available from: https://pdf.sciencedirectassets.com/271248/1-s2.0-S0141813000X01316/1-s2.0-0141813080900586/main.pdf?X-Amz-Security-Token=IQoJb3JpZ2luX2VjENj%2F%2F%2F%2F%2F%2F%2F%2F%2F%2FwEaCXVzLWVhc3QtMSJIMEYCIQDW81YTvQeiaKg%2FL1eOMe0G3pFwUaCJxg8vcs%2FeEDcaoQIhAO%2FxAyA
23. Davis M A F, Gidley M J, Morris E R, Powell D A, Rees D A. Intermolecular association in pectin solutions. Int J Biol Macromol [Internet]. 1980 Oct. 1 [cited 2021 Apr. 6]; 2(5):330-2. Available from: https://www.sciencedirect.com/science/article/pii/0141813080900598
24. Gidley M J, Morris E R, Murray E J, Powell D A, Rees D A. Evidence for two mechanisms of interchain association in calcium pectate gels. Int J Biol Macromol [Internet]. 1980 Oct. 1 [cited 2021 Apr. 6]; 2(5):332-4. Available from: https://www.sciencedirect.com/science/article/pii/0141813080900604
25. Morris G A, Foster T J, Harding S E. The effect of the degree of esterification on the hydrodynamic properties of citrus pectin. Food Hydrocoll [Internet]. 2000 May 1 [cited 2021 Apr. 6]; 14(3):227-35. Available from: https://www.sciencedirect.com/science/article/pii/S0268005X00000072?via %3Dihub
26. Vanitha T, Khan M. Role of Pectin in Food Processing and Food Packaging. In: Pectins—Extraction, Purification, Characterization and Applications [Internet]. IntechOpen; 2020 [cited 2021 Apr. 6]. Available from: https://www.intechopen.com/books/pectins-extraction-purification-characterization-and-applications/role-of-pectin-in-food-processing-and-food-packaging
27. Chan S Y, Choo W S, Young D J, Loh X J. Pectin as a rheology modifier: Recent reports on its origin, structure, commercial production and gelling mechanism. In: Loh X J, editor. RSC Polymer Chemistry Series [Internet]. royal Society of Chemistry; 2016 [cited 2021 Apr. 6]. p. 205-26. Available from: https://pubs.rsc.org
28. Moslemi M. Reviewing the recent advances in application of pectin for technical and health promotion purposes: From laboratory to market [Internet]. Vol. 254, Carbohydrate Polymers. Elsevier; 2021 [cited 2021 Apr. 6]. p. 117324. Available from: https://www.sciencedirect.com/science/article/pii/S0144861720314971

1. A method of transforming a polysaccharide into a cross-linkable polysaccharide, the method comprising:
providing a solution comprising a solubilized polysaccharide,
converting the polysaccharide into an amino-polysaccharide by replacing at least one reactive group of the polysaccharide with an amine group, thereby creating the amino-polysaccharide; and
crosslinking the amino-polysaccharide with methacrylate, thereby creating a cross-linkable polysaccharide.

2. The method of claim 1, wherein the reactive group is hydroxyl, methoxyl, methyl, ether, ester, carbonyl or carboxylic acid.

3. The method of claim 1, wherein the solubilized polysaccharide is obtained by heating a solution comprising the polysaccharide in a solvent to between about 20° C. to about 150° C. for between about 5 minutes to about an hour.

4. The method of claim 3, wherein the solvent is a polar aprotic solvent such as a mixture of dimethylacetamide and lithium chloride, dimethyl sulfoxide (DMSO), or dimethylformamide (DMF); or an ionic liquid such as 1-butyl-3-methylimidazolium chloride (BMIMCl) or 1-allyl3-methylimidazolium chloride (AMIMCl).

5. The method of claim 1, wherein the solubilized polysaccharide is obtained by cooling a solution comprising the polysaccharide in an alkali solvent, such as a mixture of NaOH and Urea, to between about −25° C. to about 10° C. for between about 5 minutes to about an hour.

6. The method of claim 1, wherein the solubilized polysaccharide is obtained by heating a solution comprising the polysaccharide, dimethylacetamide and lithium chloride to between about 50° C. to about 150° C. for between about 5 minutes to about an hour.

7. The method of claim 1, wherein the solubilized polysaccharide is obtained by:
a) centrifuging a solution comprising the polysaccharide and a solvent; and
b) repeating the centrifugation step in the presence of a highly concentrated alcohol.

8. The method of claim 1, wherein the at least one reactive group of the polysaccharide excludes hydroxyl groups directly linked to the polysaccharide backbone.

9. The method of claim 1, wherein converting the polysaccharide into an amino-polysaccharide comprises tosylation followed by amination of the at least one reactive group.

10. The method of claim 9, wherein tosylation comprises mixing the solubilized polysaccharide with triethylamine and a tosyl-containing molecule for about 24 hours at a temperature of up to about 30° C., thereby obtaining a tosyl-polysaccharide.

11. The method of claim 9, wherein tosylation comprises mixing the solubilized polysaccharide with a pyridine-ethanol solution and a tosyl-containing molecule at a low temperature for about 1 hour, thereby obtaining a tosyl-polysaccharide.

12. The method of claim 10 or 11, wherein the mixing step is followed by a step of a) filtering the mixture to obtain a solid precipitate; b) washing the solid precipitate in a highly concentrated alcohol and c) drying the solid precipitate to obtain the tosyl-polysaccharide.

13. The method of claim 9, wherein amination comprises mixing the tosyl-polysaccharide with ethylenediamine for between about 2 hours to about 48 hours.

14. The method of claim 1, wherein crosslinking the amino-polysaccharide with methacrylate comprises dissolving the amino-polysaccharide in a solvent and mixing it with a methacrylate-containing molecule at a temperature ranging from 20° C. to 60° C. (new protocols use room temperature and 50° C.).

15. The method of claim 14, wherein the mixing step comprises incubating the amino-polysaccharide with the methacrylate-containing molecule for about 1 hour to about 48 hours.

16. The method of claim 15, wherein the incubation step comprises adding a catalyst to the reaction mixture of the amino-polysaccharide and the methacrylate-containing molecule.

17. The method of claim 15, wherein the incubation step further comprises increasing the reaction temperature to about 60° C.-90° C. after the catalyst is added to the reaction mixture.

18. The method of claim 16 or 17, wherein the method further comprises:
precipitating the reaction mixture using alcohol to obtain a precipitate; and
filtering the precipitate using any filtration technique to obtain the cross-linkable polysaccharide.

19. The method of claim 18, wherein the method comprises a step of cooling the precipitate or drying the precipitate to obtain the cross-linkable polysaccharide.

20. The method of claim 1, wherein the polysaccharide is cellulose, pectin, starch, amylose, amylopectin, glycogen, gum arabic, gum ghatti, gum karaya, pullulan, β-glucans, dextran, xanthan, alginate, gellan LA, levan, hyaluronic acid or chitosan, preferably cellulose.

21. The method of claim 1, wherein the amino-polysaccharide is a pyridine-polysaccharide.

22. The method of claim 14, wherein the methacrylate-containing molecule is 2-hydroxyethyl methacrylate (HEMA), 2-hydroxypropyl methacrylate (HPMA), acrylamide (AAm), acrylic acid (AAc), N-isopropylacrylamide (NIPAm), methoxyl poly (ethylene glycol) (PEG) monoacrylate (mPEGMA or PEGMA), N,N′-methylenebis(acrylamide) (MBA), ethylene glycol diacrylate (EGDA), PEG diacrylate (PEGDA), methacrylic anhydride, glycidyl methacrylate or any other suitable methacrylate-containing molecule.

23. The method of claim 16, wherein the catalyst is triethylamine, N,N-diisopropylethylamine, N,N-diethylbenzylamine, or pyridine.

24. A method of transforming a water soluble polysaccharide into a cross-linkable polysaccharide, the method comprising:
providing a solubilized polysaccharide, and
crosslinking the solubilized polysaccharide with a methacrylate-containing molecule, thereby creating a cross-linkable polysaccharide.

25. The method of claim 24, wherein the solubilized polysaccharide is at a pH of between about 5.5 to about 10.

26. The method of claim 24, wherein crosslinking the solubilized polysaccharide comprises mixing the solubilized polysaccharide with the methacrylate-containing molecule such as methacrylic anhydride, 2-hydroxyethyl methacrylate (HEMA), 2-hydroxypropyl methacrylate (HPMA), acrylamide (AAm), acrylic acid (AAc), N-isopropylacrylamide (NIPAm), methoxyl poly (ethylene glycol) (PEG) monoacrylate (mPEGMA or PEGMA), N,N′-methylenebis(acrylamide) (MBA), ethylene glycol diacrylate (EGDA) or PEG diacrylate (PEGDA), or glycidyl methacrylate for between about 1 hour to about 72 hours at a pH of between about 8 and 8.5.

27. The method of claim 26, wherein mixing the solubilized polysaccharide with the methacylate-containing molecule is realized at a temperature of about 1° C. to about 15° C.

28. The method of claim 24, wherein crosslinking the solubilized polysaccharide comprises:
a) mixing the solubilized polysaccharide with the methacrylate-containing molecule such as methacrylic anhydride, 2-hydroxyethyl methacrylate (HEMA), 2-hydroxypropyl methacrylate (HPMA), acrylamide (AAm), acrylic acid (AAc), N-isopropylacrylamide (NIPAm), methoxyl poly (ethylene glycol) (PEG) monoacrylate (mPEGMA or PEGMA), N,N′-methylenebis(acrylamide) (MBA), ethylene glycol diacrylate (EGDA) or PEG diacrylate (PEGDA), or glycidyl methacrylate and a catalyst such as pyridine and allowing it to react for about 0.5 hour to 3 hours at a temperature ranging from 40° C. to 60° C.;
b) adding a highly concentrated alcohol to quench the reaction; and
c) isolating the methacrylate-containing molecule by filtration, thereby creating the cross-linkable polysaccharide.

29. The method of claim 28, wherein the solubilized polysaccharide is subjected to filtration to remove residual water prior to step a).

30. The method of claim 28, wherein the solubilized polysaccharide is washed and mixed with a solvent to remove residual water prior to step a).

31. The method of claim 24, wherein the water soluble polysaccharide is pectin, starch, amylose, amylopectin, glycogen, gum arabic, gum ghatti, gum karaya, pullulan, β-glucans, dextran, xanthan, alginate, gellan LA, levan or hyaluronic acid.

32. A method of transforming a cross-linkable polysaccharide into a light cross-linkable polysaccharide comprising:
providing a solution comprising the cross-linkable polysaccharide according to any one of claims 1 to 31, and
mixing the solution with about 0.1 to 0.4% w/w or % w/v of a photo-initiator in the dark, thereby making a solution comprising the light cross-linkable polysaccharide.

33. The method of claim 32, further comprising storing the solution comprising the light cross-linkable cross-linkable polysaccharide in the dark.

34. A method of transforming a light cross-linkable polysaccharide into resin comprising:
providing the solution comprising the light cross-linkable polysaccharide of claim 32 or 33, and
exposing the solution to a wavelength of between about 320 nm to about 450 nm, preferably 405 nm, for between about 5 minutes to about 30 minutes.

35. A method of preparing an intermediary methacrylated-amine molecule comprising:
mixing a methacrylate-containing molecule and an amine-containing molecule to prepare a reaction mixture;
adding a catalyst to the reaction mixture at a temperature ranging from 20° C.-50° C. to obtain the intermediary methacrylated-amine molecule.

36. The method of claim 35, wherein the methacrylate-containing molecule is 2-hydroxyethyl methacrylate (HEMA), 2-hydroxypropyl methacrylate (HPMA), acrylamide (AAm), acrylic acid (AAc), N-isopropylacrylamide (NIPAm), methoxyl poly (ethylene glycol) (PEG) monoacrylate (mPEGMA or PEGMA), N,N′-methylenebis(acrylamide) (MBA), ethylene glycol diacrylate (EGDA), PEG diacrylate (PEGDA), methacrylic anhydride, glycidyl methacrylate or any other suitable methacrylate-containing molecule.

37. The method of claim 35, wherein the amine-containing molecule is ethylenediamine, diethylenetriamine 1,3-Diaminopropane, Putrescine, Cadaverine,1,2-Dimethylethylenediamine, Hexamethylenediamine or 1,1-Dimethylethylenediamine.

38. The method of claim 35, wherein the catalyst is triethylamine, N,N-diisopropylethylamine, N,N-diethylbenzylamine, or pyridine.

39. The method of claim 35, intermediary methacrylated-amine molecule is methacrylated ethylenediamine.

40. A method of transforming a polysaccharide into a cross-linkable polysaccharide, the method comprising:
preparing a mixture comprising a tosyl-polysaccharide and an intermediary methacrylated-amine molecule; and
allowing the mixture to react for at least one hour at a temperature ranging from 70° C.-90° C., thereby obtaining the cross-linkable polysaccharide.

41. The method of claim 40, wherein the step of preparing the mixture comprises:
converting the polysaccharide to a tosyl-polysaccharide and simultaneously preparing the intermediary methacrylated-amine molecule according to claim 32;
and mixing the tosyl polysaccharide with the intermediary methacrylated-amine molecule to prepare the mixture.

42. The method of claim 40, wherein the method further comprises: precipitating the reaction mixture using alcohol to obtain a precipitate; and filtering the precipitate using any filtration technique to obtain the cross-linkable polysaccharide.

43. The method of claim 41, wherein the step of converting the polysaccharide to the tosyl polysaccharide comprises:
mixing the polysaccharide in a solubilized form with triethylamine and a tosyl-containing molecule for about 24 hours at a temperature of up to about 30° C., thereby obtaining the tosyl-polysaccharide; or
mixing the polysaccharide in a solubilized form with a pyridine-ethanol solution and a tosyl-containing molecule at a low temperature for about 1 hour, thereby obtaining the tosyl-polysaccharide.

44. A cross-linkable polysaccharide obtained according to the method of any one of claims 1-31 or 40-43.

45. A light cross-linkable polysaccharide obtained according to the method of claim 32 or 33.

46. A resin obtained according to the method of claim 34.

47. Use of the cross-linkable polysaccharide of claim 44 for preparing a light cross-linkable polysaccharide.

48. Use of the light cross-linkable polysaccharide of claim 45 for preparing a resin.

49. A method of preparing a protective coating comprising:
a) mixing component A and component B;
wherein component A is a cross-linkable polysaccharide obtained according to the method of any one of claims 1-31 or 40-43 or a light cross-linkable polysaccharide obtained according to the method of claim 32 or 33; and
wherein component B is a base formulation comprising:
i) a diacrylate;
ii) a resin; and
iii) optionally a photo-initiator;
b) curing the mixture by exposing it to UV light for a predetermined time, to obtain the protective coating.

50. The method of claim 49, wherein the mixing step comprises dispersing component B in component A at high rpm with a stir bar.

51. The method of claim 49, wherein the mixing step is followed by a step of placing the mixture in a vacuum chamber to remove bubbles.

52. The method of claim 49, wherein the curing step is carried out after applying the mixture to a surface that needs to be coated.

53. The method of claim 52, wherein the mixture is exposed to UV light ranging from 390 nm to 415 nm for about 30 seconds to 5 minutes.

54. The method of claim 49, wherein the acrylate is isobornyl acrylate or 1,6-hexanediol diacrylate; the resin is aliphatic urethane acrylate or acrylated urethane, and the optional photo-initiator is Darocur 1173.

55. Use of the protective coating prepared according to the method of claims 49-54 to prepare a nail polish coating, a varnish, a wood coating, a circuit board coating, a Teflon mold coating, an industrial coating, a plastic coating, a glass coating, a UV curable coatings, an automotive coating, a conformal coating, an optical coating, a coating for modulating hydrophobicity, or an all-purpose coating.

56. Use of the protective coating prepared according to the method of claims 49-54 for tissue engineering, tissue repair, bone tissue engineering, in-vivo or ex-vivo scaffold engineering applications, bio-ink applications, bone implants, dental treatment, dental implants, aesthetic applications, microfluidic devices and applications, microcarrier applications, medical treatment applications, wound treatment or stent applications.
